(19) United States
(12) Patent Application Publication
(10) Pub. No.: US 2008/0207704 A1
(43) Pub. Date: Aug. 28, 2008

(54) HETEROARYL-IMIDAZOLE DERIVATIVES AS CANNABINOID CB1 RECEPTOR ANTAGONISTS

(75) Inventors: Jinhwa Lee, Yongin-si (KR); Jeongmin Kim, Seoul (KR); Jong Yup Kim, Yongin-si (KR); Hee Jeong Seo, Seoul (KR); Myung Eun Jung, Seongnam-si (KR); Sung-Han Lee, Seoul (KR); Kwangwoo Ahn, Seoul (KR)

Correspondence Address: SUGHRUE MION, PLLC, 2100 PENNSYLVANIA AVENUE, N.W., SUITE 800, WASHINGTON, DC, 20037, US

(73) Assignee: THE GREEN CROSS CORPORATION, Yongin-si (KR)

(21) Appl. No.: 11/679,742

(22) Filed: Feb. 27, 2007

(51) Int. Cl. A61K 31/41; A61K 31/422; A61K 31/4245; A61K 31/427; C07D 413/04; C07D 417/04

(52) U.S. Cl. 514/363; 514/364; 514/365; 514/374; 548/137; 548/143; 548/202; 548/235

(57) ABSTRACT

A heteroaryl-imidazole compound of formula (I) or a pharmaceutically acceptable salt thereof is effective as a cannabinoid CB₁ receptor inverse agonist or antagonist, which is useful for preventing or treating obesity and obesity-related metabolic disorders. The present invention also provides a method for preparing a heteroaryl-imidazole compound of formula (I), a pharmaceutical composition containing a heteroaryl-imidazole compound of formula (I), and a method for preventing or treating obesity and obesity-related metabolic disorders.

### FIELD OF THE INVENTION

The present invention relates to a novel heteroaryl-imidazole compound which is effective as a cannabinoid CB₁ receptor inverse agonist or antagonist.

### DESCRIPTION OF THE PRIOR ART

The World Health Organization (WHO) recently reported that obesity has become a global epidemic, posing a serious threat to public health because of the increased risk of associated health problems (See Report of a WHO Consultation on Obesity: Obesity-Preventing and Managing a Global Epidemic; World Health Organization: Geneva, 1997). Obesity is characterized by excess body fat, especially visceral fat, and constitutes a pro-inflammatory state eventually leading to serious health consequences. There are growing evidences that obesity as a chronic disease cannot be cured by short-term dieting or exercise alone, but additional pharmacological treatments would lead to higher success rates.

CB₁ cannabinoid receptor belongs to G-protein-coupled receptor (GPCR) type and is coupled to inhibitory G proteins (G(i/o)) to inhibit certain adenylyl cyclase isozymes, leading to decreased cAMP production, decreased Ca²⁺ conductance, increased K⁺ conductance, and increased mitogen-activated protein kinase activity (See Di Marzo et al., Nat. Rev. Drug Discovery 2004, 3, 771-784; Rhee, M. H. et al., J. Neurochem. 1998, 71, 1525-1534). The major physiological effect of cannabinoids (in the central nervous system (CNS) and neuronal tissues) is the modulation of neurotransmitter release via activation of presynaptic CB₁ receptors located on distinct types of axon terminals throughout the brain (See Howlett, A. C. et al., Neuropharmacology 2004, 47 (Suppl. 1), 345-358).

The CB₁ receptor is mainly expressed in several brain areas including the limbic system (amygdala, hippocampus), hypothalamus, cerebral cortex, cerebellum, and basal ganglia. In the cerebellum and basal ganglia cannabinoids modulate the locomotor activity. In the limbic system, cannabinoids influence learning, memory, emotion, and motivation, and through activation of CB₁ receptors in the limbic system-hypothalamus axis, cannabinoids have an important role in the control of appetite. Moreover, lower levels of CB₁ receptors can also be found in peripheral tissues including urinary bladder, testis, prostate, GI tract, heart, lung, adrenal gland, parotid gland, bone marrow, uterus, ovary, and adipose tissue (See Cota, D. et al., J. Clin. Invest. 2003, 112, 423-431; Ravinet Trillou, C. et al., Int. J. Obes. Relat. Metab. Disord. 2004, 28, 640-648; Galiegue, S. et al., Eur. J. Biochem. 1995, 232, 54-61; Howlett, A. C. et al., Pharmacol. Rev. 2002, 54, 161-202).

Many preclinical in vitro and in vivo experiments have been shown that CB₁ receptor antagonists can influence energy homeostasis by central and peripheral mechanisms and may represent promising targets to treat diseases that are characterized by impaired energy balance. Already the first published studies with rimonabant (SR141716) in both rodents (See Arnone, M. et al., Psychopharmacology (Berlin) 1997, 132, 104-106) and primates (See Simiand, J.; Keane, M.; Keane, P. E.; Soubrie, P. Behav. Pharmacol. 1998, 9, 179-181) showed clear differentiation, i.e., marked effects on sweet food intake versus marginal effects on regular chow intake or water drinking. Many other preclinical “proof of concept” studies have been performed in the meantime with several CB agonists and antagonists to further uncover the amount and mode of contribution of cannabinergic system modulators to energy homeostasis. Almost all of those studies have been recently reviewed (See Smith, R. A. et al., IDrugs 2005, 8, 53-66).

Considering the important impact of obesity on public health and the lack of any efficient and viable drug to cure it, it is no surprise that CB₁ antagonists are currently the subject of intense studies, which were published in several reviews (See Adam, J. et al., Expert Opin. Ther. Patents, 2002, 12(10), 1475-1489; Hertzog, D. L. Expert Opin. Ther. Patents, 2004, 14(10), 1435-1452; Lange, J. H. M. et al., Drug Discov. Today, 2005, 10, 693-702; Bishop, M. J. J. Med. Chem., 2006, 49(14), 4008-4016).

### SUMMARY OF THE INVENTION

It is a primary object of the present invention to provide a novel heteroaryl-imidazole compound of formula (I) or a pharmaceutically acceptable salt thereof, which is effective as a cannabinoid CB₁ receptor inverse agonist or antagonist, useful for preventing or treating obesity and obesity-related metabolic disorders.

It is another object of the present invention to provide a method for preparing the inventive compound.

It is another object of the present invention to provide a pharmaceutical composition for preventing or treating obesity and obesity-related metabolic disorders, comprising the inventive compound as an active ingredient.

### DETAILED DESCRIPTION OF THE INVENTION

In accordance with one aspect of the present invention, there is provided a compound of formula (I) or a pharmaceutically acceptable salt thereof and a method for preparing same:

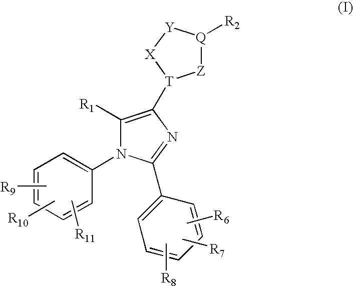

wherein:

R₁ is hydrogen, C₁₋₅ alkyl, substituted C₁₋₅ alkyl, C₂₋₄ alkenyl, substituted C₂₋₄ alkenyl, C₂₋₄ alkynyl, substituted C₂₋₄ alkynyl, or (CH₂)ₙ—C₃₋₅ carbocycle, n being 0 or 1;

R₂ is hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2;

R₃ and R₄ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or

R₃ and R₄, together with the nitrogen atom to which they are bonded, form a 4- to 10-membered saturated or unsaturated heterocyclic ring which is optionally substituted by one or more C₁₋₃ alkyl, benzyl, phenyl, C₁₋₃ alkoxy or halogen;

R₅ is phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substitutes;

R₆, R₇, R₈, R₉, R₁₀ and R₁₁ are each independently hydrogen, halogen, C₁₋₃ alkyl, C₁₋₃ alkoxy or trifluoromethyl;

X, Y and Z are each independently selected from the group consisting of —C(R₁₂)═, —O—, —N═, —N(R₁₃)— and —S— to form an aromatic heterocycle together with Q and T;

Q and T are each independently

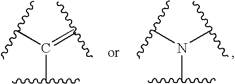

with the proviso that both Q and T can not be simultaneously

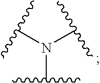

and

R₁₂ and R₁₃ are each independently hydrogen, carbocycle, substituted carbcycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, C₂₋₆ alkynyl optionally substituted by alkoxy or halogen, (CH₂)ₘ—C₃₋₆ carbocycle optionally substituted by alkoxy or halogen, or (CH₂)ₘ—R₅, m being 1 or 2, and R₅ having the same meaning as defined above.

The aromatic heterocycles formed by X, Y, Z, Q and T encompass, for example, oxazole, isoxazole, thiazole, isothiazole, triazole, oxadiazole, thiadiazole and tetrazole.

As used herein, the term “alkyl” refers to a straight or branched chain saturated hydrocarbon radical. Examples of “alkyl” as used herein include, but are not limited to, methyl, ethyl, n-propyl, isopropyl, n-butyl, isobutyl, t-butyl, n-pentyl, isopentyl and hexyl.

As used herein, the term “substituted alkyl” refers to a straight or branched chain saturated hydrocarbon radical, which is optionally substituted by one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heteroaryloxy, heterocyclic, aminosulfonyl, sulfonylamino, carboxyamide, ureido, nitro, cyano and halogen.

As used herein, the term “alkenyl” refers to a straight or branched chain hydrocarbon radical having at least one carbon-carbon double bond. Examples of “alkenyl” as used herein include, but are not limited to, ethenyl and propenyl.

As used herein, the term “substituted alkenyl” refers to a straight or branched chain hydrocarbon radical having at least one carbon-carbon double bond, which has optional substituents selected from the group consisting of C₁₋₃ alkyl optionally having one to three fluorine substituents, amino, aryl, cyano and halogen.

As used herein, the term “alkynyl” refers to a straight or branched chain hydrocarbon radical having at least one carbon-carbon triple bond. Examples of “alkynyl” as used herein include, but are not limited to, acetylenyl and 1-propynyl.

As used herein, the term “substituted alkynyl” refers to a straight or branched chain hydrocarbon radical having at least one carbon-carbon triple bond, optionally having one or more substituents selected from the group consisting of C₁₋₃ alkyl optionally having one to three fluorine substituents, amino, aryl and halogen.

As used herein, the term “halogen” refers to fluorine (F), chlorine (Cl), bromine (Br), or iodine (I).

As used herein, the term “carbocycle” refers to a non-aromatic cyclic hydrocarbon radical composed of three to seven carbon atoms. Five-to seven-membered rings may contain a double bond in the ring structure. Exemplary “carbocycle” groups include, but are not limited to, cyclopropyl, cyclobutyl, cyclopentyl, cyclopentenyl, cyclohexyl and cycloheptyl.

As used herein, the term “substituted carbocycle” refers to a non-aromatic cyclic hydrocarbon radical composed by three to seven carbon atoms, which is optionally substituted with one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heterocyclic, aminosulfonyl, sulfonylamino, carboxyamide, nitro, ureido, cyano and halogen.

As used herein, the term “aryl” refers to an optionally substituted benzene ring or refers to a ring system which may result by fusing one or more optional substituents. Exemplary optional substituents include substituted C₁₋₃ alkyl, substituted C₂₋₃ alkenyl, substituted C₂₋₃ alkynyl, heteroaryl, heterocyclic, aryl, alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, or ureido. Such a ring or ring system may be optionally fused to aryl rings (including benzene rings) optionally having one or more substituents, carbocycle rings or heterocyclic rings. Examples of “aryl” groups include, but are not limited to, phenyl, naphthyl, tetrahydronaphthyl, biphenyl, indanyl, anthracyl or phenanthryl, as well as substituted derivatives thereof.

As used herein, the term “heteroaryl” refers to an optionally substituted monocyclic five to six-membered aromatic ring containing one or more heteroatomic substitutions selected from S, SO, SO₂, O, N, or N-oxide, or refers to such an aromatic ring fused to one or more rings such as heteroaryl rings, aryl rings, heterocyclic rings, or carbocycle rings (e.g., a bicyclic or tricyclic ring system), each having optional subsituents.

Examples of optional substituents are selected from the group consisting of substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen or ureido. Examples of “heteroaryl” groups used herein include, but are not limited to, benzoimidazolyl, benzothiazolyl, benzoisothiazolyl, benzothiophenyl, benzopyrazinyl, benzotriazolyl, benzo[1,4]dioxanyl, benzofuranyl, 9H-a-carbolinyl, cinnolinyl, furanyl, furo[2,3-b]pyridinyl, imidazolyl, imidazolidinyl, imidazopyridinyl, isoxazolyl, isothiazolyl, isoquinolinyl, indolyl, indazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazolyl, pyridyl, pyrazolopyrimidinyl, pyrrolizinyl, pyridazyl, pyrazinyl, pyrimidyl, 4-oxo-1,2-dihydro-4H-pyrrolo [3,2,1-ij]-quinolin-4-yl, quinoxalinyl, quinazolinyl, quinolinyl, quinolizinyl, thiophenyl, triazolyl, triazinyl, tetrazolopyrimidinyl, triazolopyrimidinyl, tetrazolyl, thiazolyl, thiazolidinyl, and substituted versions thereof.

As used herein, the term “heterocyclic” refers to a three to seven-membered ring containing one or more heteroatomic moieties selected from S, SO, SO₂, O, N, or N-oxide, optionally substituted with one or more substituents selected from the group which includes substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, and ureido. Such a ring can be saturated or have one or more degrees of unsaturation. Such a ring may be optionally fused to one or more “heterocyclic” ring(s), aryl ring(s), heteroaryl ring(s) or carbocycle ring(s), each having optional substituents.

Examples of “heterocyclic” moieties include, but are not limited to, 1,4-dioxanyl, 1,3-dioxanyl, pyrrolidinyl, pyrrolidin-2-onyl, piperidinyl, imidazolidine-2,4-dionepiperidinyl, piperazinyl, piperazine-2,5-dionyl, morpholinyl, dihydropyranyl, dihydrocinnolinyl, 2,3-dihydrobenzo [1,4] dioxinyl, 3,4-dihydro-2H-benzo[b][1,4]-dioxepinyl, tetrahydropyranyl, 2,3-dihydrofuranyl, 2,3-dihydrobenzofuranyl, dihydroisoxazolyl, tetrahydrobenzodiazepinyl, tetrahydroquinolinyl, tetrahydrofuranyl, tetrahydronaphthyridinyl, tetrahydropurinyl, tetrahydrothiopyranyl, tetrahydrothiophenyl, tetrahydroquinoxalinyl, tetrahydropyridinyl, tetrahydrocarbolinyl, 4H-benzo[1,3]-dioxinyl, benzo[1,3]dioxonyl, 2,2-difluorobenzo-[1,3]-dioxonyl, 2,3-dihydro-phthalazine-1,4-dionyl, and isoindole-1,3-dionyl.

As used herein, the term “alkoxy” refers to the group —ORₐ, where Rₐ is alkyl as defined above. Exemplary alkoxy groups useful in the present invention include, but are not limited to, methoxy, difluoromethoxy, trifluoromethoxy, ethoxy, n-propoxy, isopropoxy, n-butoxy and t-butoxy.

As used herein the term “aralkoxy” refers to the group —ORₐR_{b}, wherein Rₐ is alkyl and R_{b} is aryl as defined above.

As used herein the term “aryloxy” refers to the group —OR_{b}, wherein R_{b} is aryl as defined above.

As used herein, the term “mercapto” refers to the group —SH.

As used herein, the term “sulfanyl” refers to the group —SR_{c}, wherein R_{c} is substituted alkyl, substituted carbocycle, aryl, heteroaryl or heterocyclic, as defined above.

As used herein, the term “sulfinyl” refers to the group —S—(O)R_{c}, wherein R_{c} is substituted alkyl, substituted carbocycle, aryl, heteroaryl or heterocyclic, as defined above.

As used herein, the term “sulfonyl” refers to the group —S(O)₂R_{c}, wherein R_{c} is substituted alkyl, substituted carbocycle, aryl, heteroaryl or heterocyclic, as defined above.

As used herein, the term “oxo” refers to the group ═O.

As used herein, the term “hydroxy” refers to the group —OH.

As used herein, the term “amino” refers to the group —NH₂. The amino group is optionally substituted by substituted alkyl, substituted carbocycle, aryl, heteroaryl or heterocyclic, as defined above.

As used herein, the term “cyano” refers to the group —CN.

As used herein, the term “aminosulfonyl” refers to the group —S(O)₂NH₂. The aminosulfonyl group is optionally substituted by substituted alkyl, substituted carbocycle, aryl, heteroaryl or heterocyclic, as defined above.

As used herein, the term “sulfonylamino” refers to the group —NHS(O)₂R_{c} wherein R_{c} is substituted alkyl, substituted carbocycle, aryl, heteroaryl or heterocyclic, as defined above.

As used herein, the term “carboxyamide” refers to the group —NHC(O)R_{c} wherein R_{c} is substituted alkyl, substituted carbocycle, aryl, heteroaryl or heterocyclic, as defined above.

As used herein, the term “carboxy” refers to the group —C(O)OH. The carboxy group is optionally substituted by substituted alkyl, substituted carbocycle, aryl, heteroaryl or heterocyclic, as defined above.

As used herein, the term “aminocarbonyl” refers to the group —C(O)NH₂. The aminocarbonyl group is optionally substituted by substituted alkyl, substituted carbocycle, aryl, heteroaryl or heterocyclic, as defined above.

As used herein, the term “ureido” refers to the group —NHC(O)NHR_{d} wherein R_{d} is hydrogen, alkyl, carbocycle or aryl as defined above.

As used herein, the term “guanidino” refers to the group —NHC(═NH)NH₂.

As used herein, the term “acyl” refers to the group —C(O)Rₑ, wherein Rₑ is alkyl, carbocycle, or heterocyclic as defined herein.

As used herein, the term “aroyl” refers to the group —C(O)R_{b}, wherein R_{b} is aryl as defined herein.

As used herein, the term “heteroaroyl” refers to the group —C(O)R_{f}, wherein R_{f} is heteroaryl as defined herein.

As used herein, the term “acyloxy” refers to the group —OC(O)Rₑ, wherein Rₑ is alkyl, carbocycle, or heterocyclic as defined herein.

As used herein, the term “aroyloxy” refers to the group —OC(O)R_{b}, wherein R_{b} is aryl as defined herein.

As used herein, the term “heteroaroyloxy” refers to the group —OC(O)R_{f}, wherein R_{f} is heteroaryl as defined herein.

It is to be understood that the present invention also includes a pharmaceutically acceptable salt and an addition salt of the inventive compound, such as a hydrochloride, hydrobromide or trifluoroacetate addition salt and a sodium, potassium and magnesium salt.

The compounds of the present invention may contain one or more asymmetric carbon atoms and may exist in racemic and optically active forms. All of these compounds and diastereomers are incorporated within the scope of the present invention.

One embodiment of the present invention is to provide a compound of formula (Ia) or a pharmaceutically acceptable salt thereof:

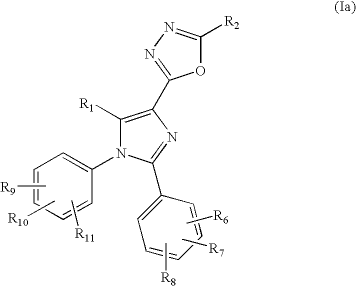

wherein, R₁, R₂, R₆, R₇, R₈, R₉, R₁₀ and R₁₁ have the same meanings as defined above.

Another embodiment of the present invention is to provide a compound of formula (Ib) or a pharmaceutically acceptable salt thereof:

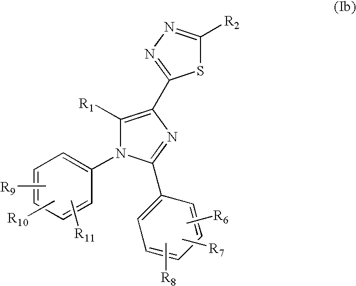

wherein, R₁, R₂, R₆, R₇, R₈, R₉, R₁₀ and R₁₁ have the same meanings as defined above.

A further embodiment of the present invention is to provide a compound of formula (Ic) or a pharmaceutically acceptable salt thereof:

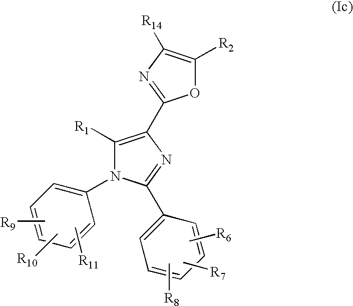

wherein R₁, R₂, R₆, R₇, R₈, R₉, R₁₀ and R₁₁ have the same meanings as defined above, and R₁₄ has the same meaning as defined for R₂. Alternatively, R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

A still another embodiment of the present invention is to provide a compound of formula (Id) or a pharmaceutically acceptable salt thereof:

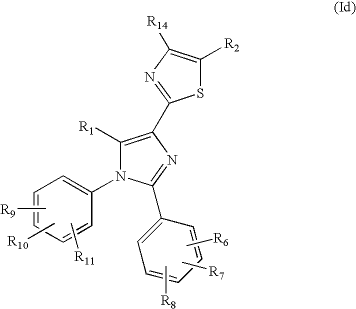

wherein, R₁, R₂, R₆, R₇, R₈, R₉, R₁₀ and R₁₁ have the same meanings as defined above, and R₁₄ has the same meaning as defined for R₂. Alternatively, R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

The compounds of the present invention and the preparation thereof will be better understood in connection with the following synthetic schemes, which are merely illustrative of the methods by which the compounds of the invention may be prepared and are not intended to limit the scope of the invention as defined in the appended claims.

The compound of formula (Ia) may be prepared by (i) reacting a carboxylic acid derivative (6) with a hydrazide compound (7) in the presence of a coupling reagent, e.g. EDCI, DMAP, and (ii) cyclizing the resulting product (8) using a dehydrating agent to obtain an 1,3,4-oxadiazole, as shown in Reaction Scheme 1. The cyclization may be conducted using Burgess reagent as a dehydrating agent while applying microwave irradiation thereon (See Leber, J. D. et al., WO 2005/032550), or using triphenylphosphine with carbon tetrachloride and a base such as triethylamine in a suitable solvent such as acetonitrile and THF, or using POCl₃ in an appropriate solvent such as acetonitrile.

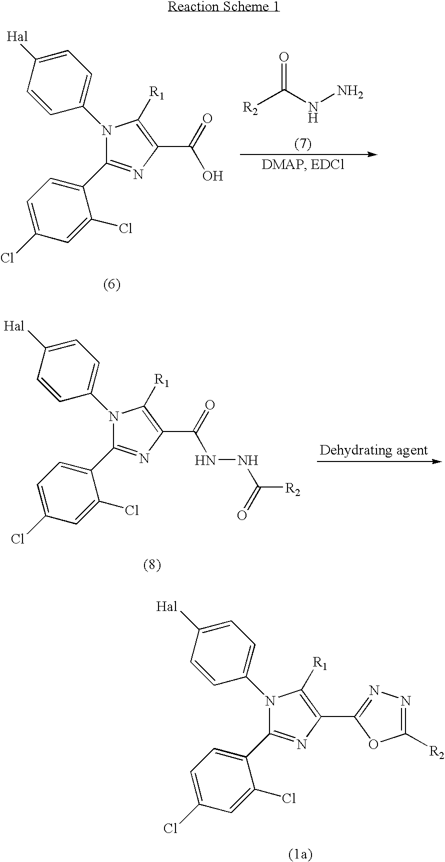

wherein, R₁ and R₂ have the same meanings as defined above.

The carboxylic acid derivative (6) used as a starting material in preparing the compound of formula (Ia) may be prepared by a conventional method, e.g., by reacting a benzonitrile derivative (1) with an aniline derivative (2) such as 4-chloroaniline using a non-nucleophilic base such as sodium bis(trimethylsilyl)amide (NaHMDS) to produce a corresponding arylbenzamidine (3), subsequently reacting the resulting arylbenzamidine (3) with ethyl 3-bromo-2-oxobutanoate (4) to provide an intermediate ethyl 1,2-diaryl-5-methyl-1H-imidazole-4-carboxylate (5), then transforming the intermediate (5) into an acid form (6) using an alkaline agent such as potassium hydroxide or lithium hydroxide, followed by acidification (See Lange, J. H. M. et al., J. Med. Chem. 2005, 48, 1823), as shown in Reaction Scheme 2.

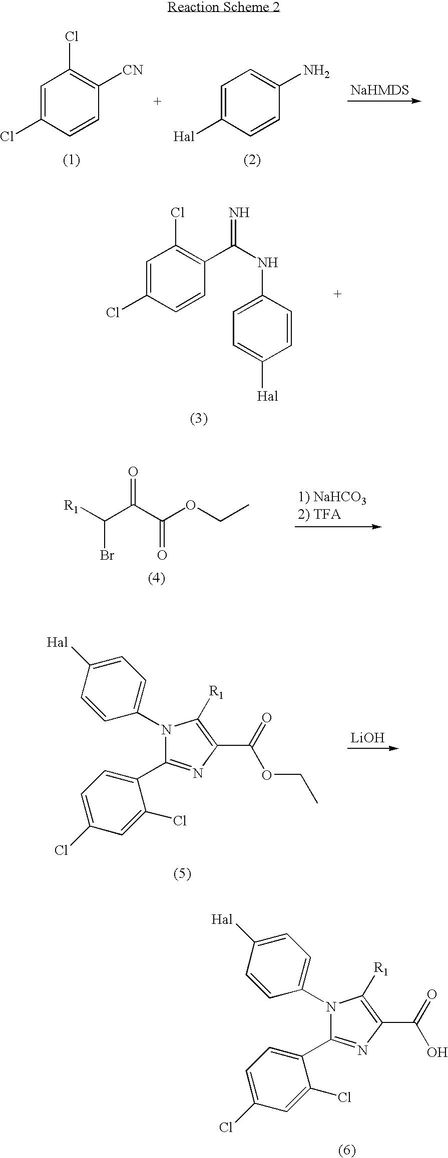

wherein, R₁ has the same meaning as defined above.

The hydrazide compound (7) which may be used in preparing the compound of formula (Ia) may be prepared by treating an ester or a carboxylic acid with hydrazine, as shown in Reaction Scheme 3.

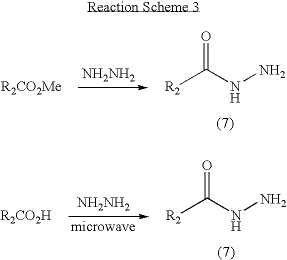

wherein, R₂ has the same meaning as defined above.

The compound of formula (Ib) may be prepared by (i) reacting a carboxylic acid derivative (6) with a hydrazide compound (7) in the presence of a coupling reagent, e.g., EDCI, DMAP, and (ii) cyclizing the resulting product (8) using Lawesson's reagent, which can be conducted with microwave irradiation (See Kiryanov, A. A., Sampson, P., Seed, A. J., J. Org. Chem. 2001, 665, 7925-7929), as shown in Reaction Scheme 4.

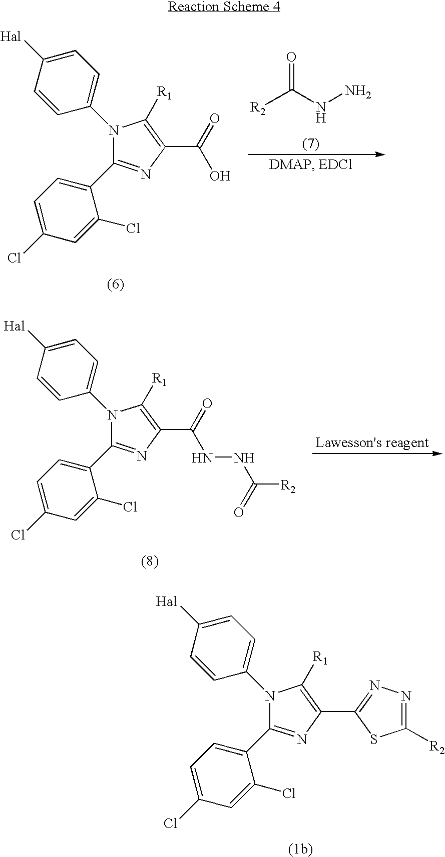

wherein, R₁ and R₂ have the same meanings as defined above.

The compound of formula (Ic) may be prepared by (i) reacting a carboxylic acid derivative (6) with a salt of an aminoketone compound (9) in the presence of a coupling reagent, e.g. EDCI, DMAP, and (ii) cyclizing the resulting product (10) using a dehydrating agent, to obtain an oxazole compound, as shown in Reaction Scheme 5. The cyclization may be conducted using Burgess reagent as a dehydrating agent while applying microwave irradiation thereon.

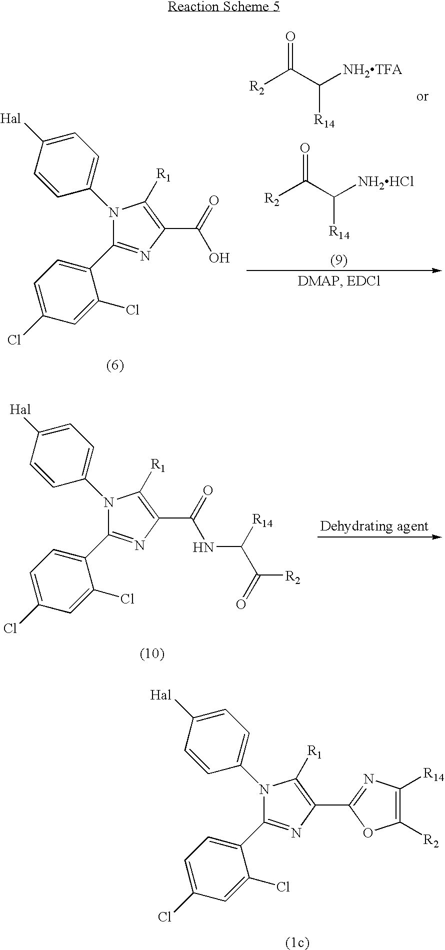

wherein R₁, R₂ and R₁₄ have the same meanings as defined above.

The salt of the aminoketone compound (9) which may be used in preparing the compound of formula (Ic) may be prepared as shown in Reaction Scheme 6. For example, the starting N-Boc protected aminoacid (11) is converted into the corresponding Weinreb amide (12) using N,O-dimethylhydroxylamine hydrochloride in the presence of a coupling reagent such as EDCI, HOBt, NMM in an appropriate solvent such as DCM or DMF. The Weinreb amide (12) may be transformed into a corresponding ketone (13) by action of a Grignard reagent in an appropriate solvent such as ether or THF under N₂ atmosphere. Then, final deprotection of Boc group using either TFA or HCl may provide the aminoketone compound (9) in a salt form, respectively.

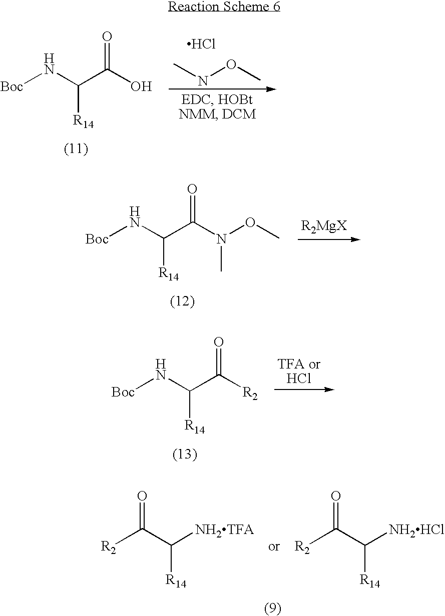

wherein R₂ and R₁₄ have the same meanings as defined above.

The compound of formula (Id) may be prepared by (i) reacting a carboxylic acid derivative (6) with a salt of an aminoketone compound (9) in the presence of a coupling reagent, e.g. EDCI, DMAP, and (ii) cyclizing the resulting product (10) using Lawesson's reagent, which can be conducted with microwave irradiation (See Kiryanov, A. A., Sampson, P., Seed, A. J., J. Org. Chem. 2001, 665, 7925-7929), as shown in Reaction Scheme 7.

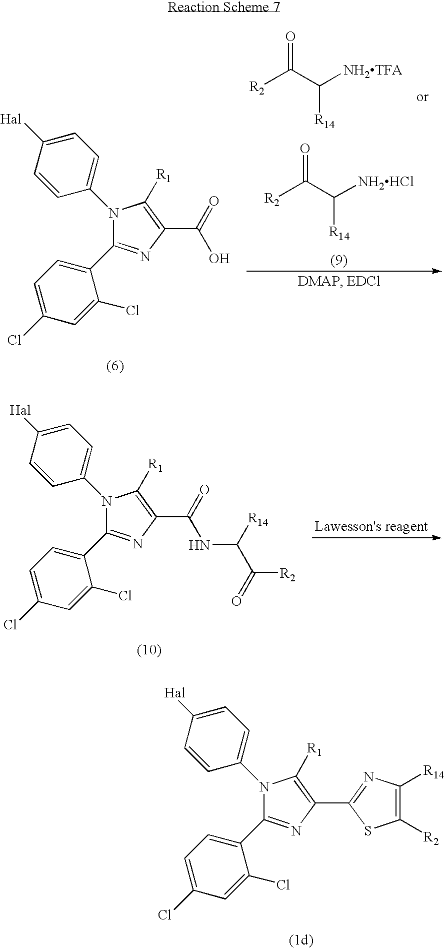

wherein R₁, R₂ and R₁₄ have the same meanings as defined above.

The inventive heteroaryl-imidazole compound of formula (I) is effective as a cannabinoid CB₁ receptor inverse agonist or antagonist, thereby preventing or treating obesity and obesity-related metabolic disorders.

Accordingly, the present invention provides a pharmaceutical composition for preventing or treating obesity and obesity-related metabolic disorders, which comprises the compound of formula (I) or a pharmaceutically acceptable salt thereof as an active ingredient and a pharmaceutically acceptable carrier.

Also, the present invention provides a method for inhibiting cannabinoid CB₁ receptor in a mammal, which comprises administering the compound of formula (I) or a pharmaceutically acceptable salt thereof to the mammal.

Further, the present invention provides a method for preventing or treating obesity and obesity-related metabolic disorders in a mammal, which comprises administering the compound of formula (I) or a pharmaceutically acceptable salt thereof to the mammal.

As used herein, the term “obesity-related metabolic disorders” refers to chronic diseases that require treatment to reduce the excessive health risks associated with obesity and exemplary disorders include type 2 diabetes mellitus, cardiovascular and hypertension, hyperlipidaemia, fibrinolytic abnormalities.

The pharmaceutical composition may be administered orally, intramuscularly or subcutaneously. The formulation for oral administration may take various forms such as a syrup, tablet, capsule, cream and lozenge. A syrup formulation will generally contain a suspension or solution of the compound or its salt in a liquid carrier, e.g., ethanol, peanut oil, olive oil, glycerine or water, optionally with a flavoring or coloring agent. When the composition is formulated in the form of a tablet, any one of pharmaceutical carriers routinely used for preparing solid formulations may be used. Examples of such carriers include magnesium stearate, terra alba, talc, gelatin, acacia, stearic acid, starch, lactose and sucrose. When the composition is formulated in the form of a capsule, any of the routine encapsulation procedures may be employed, e.g., using the aforementioned carriers in a hard gelatin capsule shell. When the composition is formulated in the form of a soft gelatin shell capsule, any of the pharmaceutical carrier routinely used for preparing dispersions or suspensions may be prepared using an aqueous gum, cellulose, silicate or oil. The formulation for intramuscular or subcutaneous administration may take a liquid form such as a solution, suspension and emulsion which includes aqueous solvents such as water, physiological saline and Ringer's solution; or lipophilic solvents such as fatty oil, sesame oil, corn oil and synthetic fatty acid ester.

Preferably the composition is formulated in a specific dosage form for a particular patient.

Each dosage unit for oral administration contains suitably from 0.1 mg to 500 mg/Kg, and preferably from 1 mg to 100 mg/Kg of the compound of formula (I) or its pharmaceutically acceptable salt.

The suitable daily dosage for oral administration is about 0.01 mg/Kg to 40 mg/Kg of the compound of formula (I) or its pharmaceutically acceptable salt, may be administered 1 to 6 times a day, depending on the patient's condition.

The present invention is further described and illustrated in Examples provided below, which are, however, not intended to limit the scope of the present invention.

### EXAMPLE

As used herein the symbols and conventions used describing the processes, schemes and examples of the present invention are consistent with those used in the contemporary scientific literature, for example, the Journal of the American Chemical Society or the Journal of Biological Chemistry. Unless otherwise noted, all starting materials were obtained from commercial suppliers and used without further purification.

Hz (Hertz) TLC (thin layer chromatography)

Tᵣ (retention time) RP (reverse phase)

MeOH (methanol) i-PrOH (isopropanol)

TFA (trifluoroacetic acid) TEA (triethylamine)

EtOH (ethanol) THF (tetrahydrofuran)

DMSO (dimethylsulfoxide) EtOAc (ethyl acetate)

DCM (dichloromethane) HOAc (acetic acid)

DMF (N,N-dimethylformamide) Ac (acetyl)

CDI (1,1-carbonyldiimidazole) Bn (benzyl)

HOSu (N-hydroxysuccinimide)

HOBT (1-hydroxybenzotriazole)

Boc (tert-butyloxycarbonyl)

mCPBA (meta-chloroperbenzoic acid)

FMOC (9-fluorenylmethoxycarbonyl)

DCC (dicyclohexylcarbodiimide)

Cbz (benzyloxycarbonyl)

NMM (N-methyl morpholine)

HOAt (1-hydroxy-7-azabenzotriazole)

TBAF (tetra-n-butylammonium fluoride)

THP (tetrahydro-2H-pyran-2-yl)

DMAP (4-dimethylaminopyridine)

HPLC (high pressure liquid chromatography)

BOP (bis(2-oxo-3-oxazolidinyl)phosphinic chloride);

EDCI (1-ethyl-3-[3-dimethylaminopropyl]carbodiimide hydrochloride)

HBTU (O-Benzotriazole 1-yl-N,N,N′,N′-tetramethyluronium hexafluorophosphate)

All references to ether are to diethyl ether; brine refers to a saturated aqueous solution of NaCl. Unless otherwise indicated, all temperatures are expressed in □ (degrees Centigrade). All reactions are conducted under an inert atmosphere at room temperature unless otherwise noted, and all solvents are of the highest available purity unless otherwise indicated.

Microwave reaction was conducted with a Biotage microwave reactor.

¹H NMR spectra were recorded on either a Jeol ECX-400, or a Jeol JNM-LA300 spectrometer. Chemical shifts were expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Mass spectra were obtained with either a Micromass, Quattro LC Triple Quadruple Tandem Mass Spectometer, ESI or Agilent, 1100 LC/MSD, ESI.

For preparative HPLC, ca 100 mg of a product was injected in 1 mL of DMSO onto a SunFire™ Prep C18 OBD 5 μm 19×100 mm Column with a 10 min gradient from 10% CH₃CN to 90% CH₃CN in H₂O. Flash chromatography was carried using Merck silica gel 60 (230-400 mesh). Most of the reactions were monitored by thin-layer chromatography on 0.25 mm E. Merck silica gel plates (60F-254), visualized with UV light using a 5% ethanolic phosphomolybdic acid or p-anisaldehyde solution.

The following synthetic schemes are merely illustrative of the methods by which the compounds of the invention may be prepared and are not intended to limit the scope of the invention as defined in the appended claims.

### Preparation of 1,3,4-oxadiazole (Formula (Ia))

### Example 1

### 2-tert-Butyl-5-(1-(4-chlorophenyl)-2-(2,4-dichlorophenyl)-5-methyl-1H-imidazol-4-yl)-1,3,4-oxadiazole

### Step 1:

### 1-(4-chlorophenyl)-2-(2,4-dichlorophenyl)-5-methyl-N′-pivaloyl-1H-imidazole-4-carbohydrazide

1-(4-Chlorophenyl)-2-(2,4-dichlorophenyl)-5-methyl-1H-imidazole-4-carboxylic acid (0.20 g, 0.524 mmol), pivalohydrazide (73 mg, 0.629 mmol), EDCI (0.24 g, 1.26 mmol) and HOBt (85 mg, 0.629 mmol) were dissolved in DCM (5 ml), to which NMM (0.32 g, 3.15 mmol) was added in one portion at room temperature. The reaction mixture was stirred at room temperature for 18 hr. The organic layer was collected and evaporated under a vacuum. The crude mixture was further purified by preparative HPLC, to obtain 102 mg (0.213 mmol, 41%) of the title compound as a yellow solid.

¹H NMR (400 MHz, CDCl₃)) δ 8.14 (br, s, 1H), 7.36-7.34 (m, 2H), 7.33 (d, J=1.8 Hz, 1H), 7.28 (d, J=8.2 Hz, 1H), 7.24 (dd, J=8.2, 1.8 Hz, 1H), 7.05-7.03 (m, 2H), 2.46 (s, 3H), 1.31 (s, 9H).

MH+479.

### Step 2:

### 2-tert-Butyl-5-(1-(4-chlorophenyl)-2-(2,4-dichlorophenyl)-5-methyl-1H-imidazol-4-yl)-1,3,4-oxadiazole

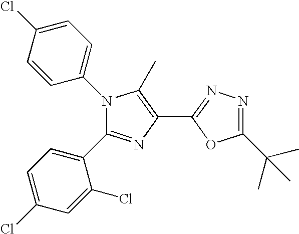

1-(4-Chlorophenyl)-2-(2,4-dichlorophenyl)-5-methyl-N′-pivaloyl-1H-imidazole-4-carbohydrazide (40 mg, 0.083 mmol) obtained in Step 1 was added to a microwave reactor containing Burgess reagent (60 mg, 0.250 mmol) in THF (1 mL). The microwave reactor was capped and the mixture was irradiated at 160□ for 30 min. The reaction product was purified by preparative HPLC to provide the title compound (14.2 mg, 0.031 mmol, 37%) as a yellow solid.

¹H NMR (400 MHz, CDCl₃) δ 7.38-7.36 (m, 3H), 7.32 (d, J=2.3 Hz, 1H), 7.25 (dd, J=8.2, 2.3 Hz, 1H), 7.10-7.08 (m, 2H), 2.53 (s, 3H), 1.50 (s, 9H).

MH+461.

The following compounds of Examples 2 to 21 were obtained by repeating the procedure of Example 1.

### Example 2

### 2-(1-(4-Chlorophenyl)-2-(2,4-dichlorophenyl)-5-methyl-1H-imidazol-4-yl)-5-cyclohexyl-1,3,4-oxadiazole

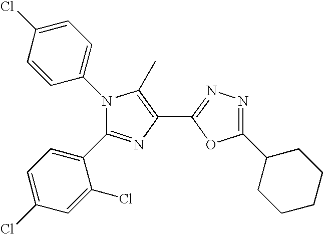

¹H NMR (400 MHz, CDCl₃) δ 7.39-7.34 (m, 3H), 7.32 (d, J=2.3 Hz, 1H), 7.26-7.23 (m, 1H), 7.09 (d, J=8.7 Hz, 2H), 3.03-2.95 (m, 1H), 2.54 (s, 3H), 2.16-2.12 (m, 2H), 1.88-1.84 (m, 2H), 1.77-1.67 (m, 3H), 1.44-1.25 (m, 3H).

MH+487.

### Example 3

### 2-(1-(4-Chlorophenyl)-2-(2,4-dichlorophenyl)-5-methyl-1H-imidazol-4-yl)-5-cyclopentyl-1,3,4-oxadiazole

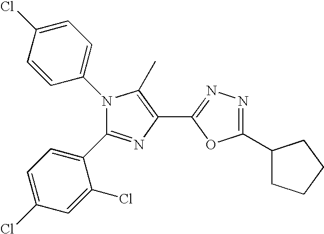

¹H NMR (400 MHz, CDCl₃) δ 7.39-7.35 (m, 3H), 7.32 (d, J=1.8 Hz, 1H), 7.25 (dd, J=8.2, 2.3 Hz, 1H), 7.10 (d, J=8.7 Hz, 2H), 3.43-3.35 (m, 1H), 2.54 (s, 3H), 2.18-2.10 (m, 2H), 2.08-1.99 (m, 2H), 1.89-1.79 (m, 2H), 1.75-1.66 (m, 2H).

MH+473.

### Example 4

### 2-(1-(4-Chlorophenyl)-2-(2,4-dichlorophenyl)-5-methyl-1H-imidazol-4-yl)-5-cyclobutyl-1,3,4-oxadiazole

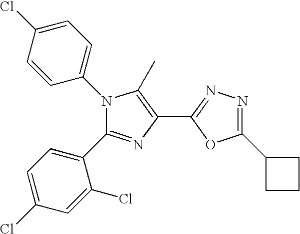

¹H NMR (400 MHz, CDCl₃) δ 7.39-7.34 (m, 3H), 7.33 (d, J=1.8 Hz, 1H), 7.26-7.23 (dd, J=8.2, 1.8 Hz, 1H), 7.10 (d, J=8.7 Hz, 2H), 3.85-3.77 (m, 1H), 2.59-2.52 (m, 5H), 2.48-2.40 (m, 2H), 2.18-1.99 (m, 2H).

MH+459.

### Example 5

### 2-(1-(4-Chlorophenyl)-2-(2,4-dichlorophenyl)-5-methyl-1H-imidazol-4-yl)-5-cycloheptyl-1,3,4-oxadiazole

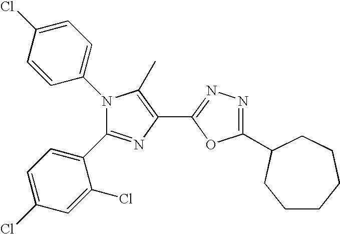

¹H NMR (400 MHz, CDCl₃) δ 7.39-7.34 (m, 3H), 7.32 (d, J=2.3 Hz, 1H), 7.24 (dd, J=8.2, 2.3 Hz, 1H), 7.11-7.07 (m, 2H), 3.22-3.15 (m, 1H), 2.54 (s, 3H), 2.20-2.14 (m, 2H), 1.99-1.89 (m, 2H), 1.85-1.79 (m, 2H), 1.68-1.53 (m, 6H).

MH+501.

### Example 6

### 2-(1-(4-Chlorophenyl)-2-(2,4-dichlorophenyl)-5-methyl-1H-imidazol-4-yl)-5-(hexan-2-yl)-1,3,4-oxadiazole

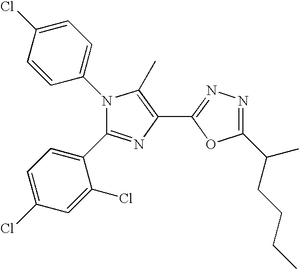

¹H NMR (400 MHz, CDCl₃) δ 7.39-7.35 (m, 3H), 7.33 (d, J=1.8 Hz, 1H), 7.25 (dd, J=8.2, 1.8 Hz, 1H), 7.11-7.07 (m, 2H), 3.21-3.12 (m, 1H), 2.54 (s, 3H), 1.97-1.88 (m, 1H), 1.73-1.64 (m, 1H), 1.42 (d, J=6.9 Hz, 3H), 1.37-1.28(m, 4H), 0.89 (t, J=7.3 Hz, 3H).

MH+489.

### Example 7

### 2-(1-(4-Chlorophenyl)-2-(2,4-dichlorophenyl)-5-methyl-1H-imidazol-4-yl)-5-(pentan-2-yl)-1,3,4-oxadiazole

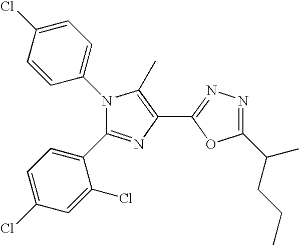

¹H NMR (400 MHz, CDCl₃) δ 7.39-7.35 (m, 3H), 7.32 (d, J=2.3 Hz, 1H), 7.25 (dd, J=8.2, 2.3 Hz, 1H), 7.11-7.07 (m, 2H), 3.23-3.14 (m, 1H), 2.54 (s, 3H), 1.96-1.87 (m, 1H), 1.71-1.61 (m, 1H), 1.44-1.32 (m, 5H), 0.92 (t, J=7.3 Hz, 3H).

MH+475.

### Example 8

### 2-sec-Butyl-5-(1-(4-chlorophenyl)-2-(2,4-dichlorophenyl)-5-methyl-1H-imidazol-4-yl)-1,3,4-oxadiazole

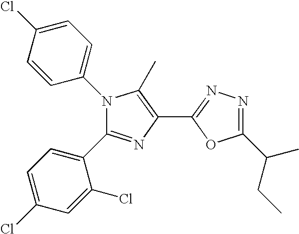

¹H NMR (400 MHz, CDCl₃) δ 7.39-7.35 (m, 3H), 7.33 (d, J=1.8 Hz, 1H), 7.25 (dd, J=8.2, 1.8 Hz, 1H), 7.11-7.07 (m, 2H), 3.15-3.06 (m, 1H), 2.54 (s, 3H), 2.01-1.90 (m, 1H), 1.80-1.69 (m, 1H), 1.43 (d, J=7.3 Hz, 3H), 0.97 (t, J=7.3 Hz, 3H).

MH+461.

### Example 9

### 2-(1-(4-Chlorophenyl)-2-(2,4-dichlorophenyl)-5-methyl-1H-imidazol-4-yl)-5-isopropyl-1,3,4-oxadiazole

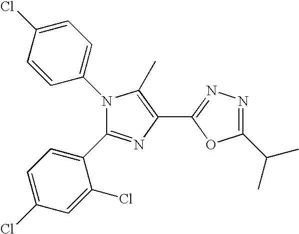

¹H NMR (400 MHz, CDCl₃) δ 7.39-7.35 (m, 3H), 7.33 (d, J=1.8 Hz, 1H), 7.25 (dd, J=8.2, 2.3 Hz, 1H), 7.11-7.08 (m, 2H), 3.33-3.25 (m, 1H), 2.54 (s, 3H), 1.46 (d, J=6.9 Hz, 6H).

MH+447.

### Example 10

### 2-(1-(4-Chlorophenyl)-2-(2,4-dichlorophenyl)-5-methyl-1H-imidazol-4-yl)-5-(pentan-3-yl)-1,3,4-oxadiazole

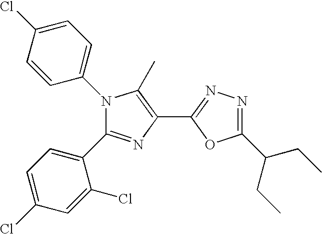

¹H NMR (400 MHz, CDCl₃) δ 7.39-7.32 (m, 4H), 7.27-7.24 (m, 1H), 7.10-7.08 (m, 2H), 2.97-2.93 (m, 1H), 2.55 (s, 3H), 1.93-1.76 (m, 4H), 0.93 (t, J=7.2 Hz, 6H).

MH+475.

### Example 11

### 2-(1-(4-Chlorophenyl)-2-(2,4-dichlorophenyl)-5-methyl-1H-imidazol-4-yl)-5-(1-phenylethyl)-1,3,4-oxadiazole

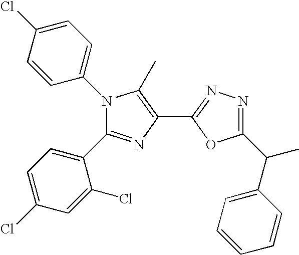

¹H NMR (400 MHz, CDCl₃) δ 7.38-7.33 (m, 5H), 7.32-7.30 (m, 3H), 7.27-7.22 (m, 2H), 7.09-7.05 (m, 2H), 4.44 (q, J=7.3 Hz, 1H), 2.51 (s, 3H), 1.82 (d, J=7.3 Hz, 3H).

MH+509.

### Example 12

### 2-(1-(4-Chlorophenyl)-2-(2,4-dichlorophenyl)-5-ethyl-1H-imidazol-4-yl)-5-cyclohexyl-1,3,4-oxadiazole

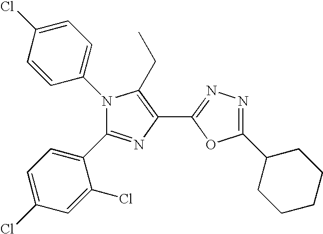

¹H NMR (400 MHz, CDCl₃) δ 7.39-7.32 (m, 4H), 7.23 (dd, J=8.2, 1.8 Hz, 1H), 7.15-7.12 (m, 2H), 3.03-2.97 (m, 3H), 2.15 (d, J=11 Hz, 2H), 1.87-1.84 (m, 2H), 1.76-1.67 (m, 3H), 1.44-1.25 (m, 3H), 1.10 (t, J=7.8 Hz, 3H).

MH+501.

### Example 13

### 2-(1-(4-Chlorophenyl)-2-(2,4-dichlorophenyl)-5-ethyl-1H-imidazol-4-yl)-5-cyclopentyl-1,3,4-oxadiazole

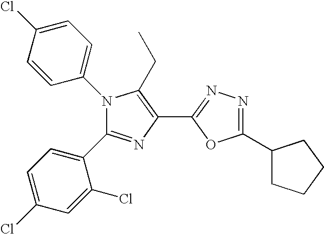

¹H NMR (400 MHz, CDCl₃) δ 7.39-7.32 (m, 4H), 7.23 (dd, J=8.2, 1.8 Hz, 1H), 7.13 (d, J=8.2 Hz, 2H), 3.43-3.34 (m, 1H), 3.00 (q, J=7.3 Hz, 2H), 2.19-2.00 (m, 4H), 1.89-1.79 (m, 2H), 1.75-1.67 (m, 2H), 1.10 (t, J=7.3 Hz, 3H).

MH+487.

### Example 14

### 2-(1-(4-Chlorophenyl)-2-(2,4-dichlorophenyl)-5-ethyl-1H-imidazol-4-yl)-5-cyclobutyl-1,3,4-oxadiazole

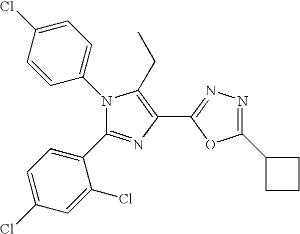

¹H NMR (400 MHz, CDCl₃) δ 7.40-7.32 (m, 4H), 7.23 (dd, J=8.2, 1.8 Hz, 1H), 7.16-7.12 (m, 2H), 3.86-3.77 (m, 1H), 3.01 (q, J=7.3 Hz, 2H), 2.62-2.52 (m, 2H), 2.47-2.39 (m, 2H), 2.16-2.01 (m, 2H), 1.11 (t, J=7.3 Hz, 3H).

MH+473.

### Example 15

### 2-tert-Butyl-5-(1-(4-chlorophenyl)-2-(2,4-dichlorophenyl)-5-ethyl-1H-imidazol-4-yl)-1,3,4-oxadiazole

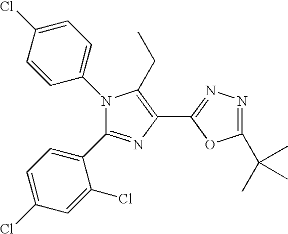

¹H NMR (300 MHz, CDCl₃) δ 7.36-7.30 (m, 4H), 7.27-7.24 (m, 1H), 7.14-7.11 (m, 2H), 2.98 (q, J=7.3 Hz, 2H), 1.50 (s, 9H), 1.11 (t, J=7.3 Hz, 3H).

MH+475.

### Example 16

### 2-(1-(4-Chlorophenyl)-2-(2,4-dichlorophenyl)-5-ethyl-1H-imidazol-4-yl)-5-(hexan-2-yl)-1,3,4-oxadiazole

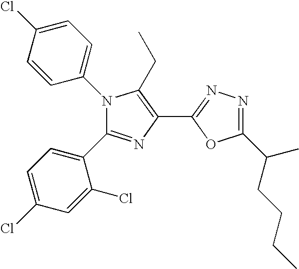

¹H NMR (300 MHz, CDCl₃) δ 7.40-7.32 (m, 4H), 7.24 (dd, J=8.2, 2.2 Hz, 1H), 7.15-7.12 (m, 2H), 3.20-3.11 (m, 1H), 3.00 (q, J=7.3 Hz, 2H), 1.97-1.90 (m, 1H), 1.71-1.61 (m, 1H), 1.42 (d, J=7.0 Hz, 3H), 1.35-1.32 (m, 4H), 1.11 (t, J=7.3 Hz, 3H), 0.89 (t, J=6.8 Hz, 3H).

MH+503.

### Example 17

### 2-(1-(4-Chlorophenyl)-2-(2,4-dichlorophenyl)-5-ethyl-1H-imidazol-4-yl)-5-(pentan-2-yl)-1,3,4-oxadiazole

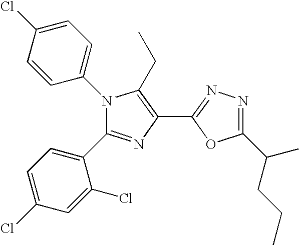

¹H NMR (400 MHz, CDCl₃) δ 7.39-7.32 (m, 4H), 7.24 (dd, J=8.4, 2.0 Hz, 1H), 7.14-7.11 (m, 2H), 3.21-3.16 (m, 1H), 3.00 (q, J=7.6 Hz, 2H), 1.93-1.89 (m, 1H), 1.69-1.60 (m, 1H), 1.41-1.35 (m, 5H), 1.11 (t, J=7.6 Hz, 3H), 0.92 (t, J=7.2 Hz, 3H).

MH+489.

### Example 18

### 2-sec-Butyl-5-(1-(4-chlorophenyl)-2-(2,4-dichlorophenyl)-5-ethyl-1H-imidazol-4-yl)-1,3,4-oxadiazole

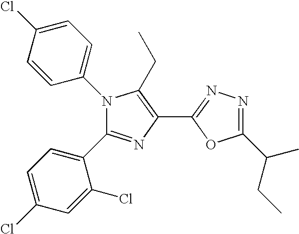

¹H NMR (400 MHz, CDCl₃) δ 7.40-7.34 (m, 3H), 7.32 (d, J=2.0 Hz, 1H), 7.24 (dd, J=8.4, 2.0 Hz, 1H), 7.15-7.12 (m, 2H), 3.13-3.08 (m, 1H), 3.01 (q, J=7.6 Hz, 2H), 1.99-1.92 (m, 1H), 1.78-1.71 (m, 1H), 1.43 (d, J=7.2 Hz, 3H), 1.11 (t, J=7.2 Hz, 3H), 0.97 (t, J=7.6 Hz, 3H).

MH+475.

### Example 19

### 2-(1-(4-Bromophenyl)-2-(2,4-dichlorophenyl)-5-methyl-1H-imidazol-4-yl)-5-cyclohexyl-1,3,4-oxadiazole

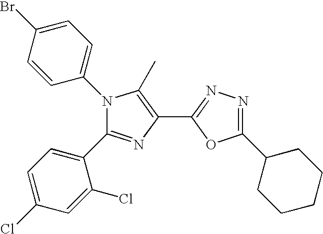

¹H NMR (400 MHz, CDCl₃) δ 7.55-7.52 (m, 2H), 7.36 (d, J=8.0 Hz, 1H), 7.33 (d, J=1.6 Hz, 1H), 7.27-7.24 (m, 1H), 7.04-7.02 (m, 2H), 3.03-2.95 (m, 1H), 2.54 (s, 3H), 2.16-2.13 (m, 2H), 1.88-1.84 (m, 2H), 1.77-1.67 (m, 3H), 1.45-1.26 (m, 3H).

MH+531.

### Example 20

### 2-(1-(4-Bromophenyl)-2-(2,4-dichlorophenyl)-5-methyl-1H-imidazol-4-yl)-5-tert-butyl-1,3,4-oxadiazole

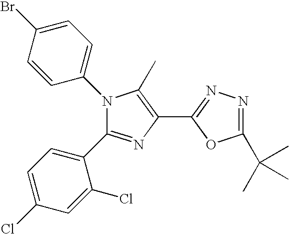

¹H NMR (400 MHz, CDCl₃) δ 7.54-7.51 (m, 2H), 7.39-7.36 (m, 1H), 7.33-7.32 (m, 1H), 7.27-7.24 (m, 1H), 7.04-7.01 (m, 2H), 2.53 (s, 3H), 1.50 (s, 9H).

MH+505.

### Example 21

### 2-(1-(4-Bromophenyl)-2-(2,4-dichlorophenyl)-5-ethyl-1H-imidazol-4-yl)-5-tert-butyl-1,3,4-oxadiazole

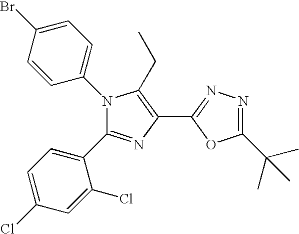

¹H NMR (400 MHz, CDCl₃) δ 7.54-7.52 (m, 2H), 7.37-7.35 (m, 1H), 7.32 (m, 1H), 7.24 (dd, J=8.4, 2.0 Hz, 1H), 7.08-7.05 (m, 2H), 2.99 (q, J=7.2 Hz, 2H), 1.50 (s, 9H), 1.11 (t, J=7.6 Hz, 3H).

MH+519.

### Preparation of 1,3,4-thiadiazole (Formula (Ib))

### Example 22

### 2-tert-Butyl-5-(1-(4-chlorophenyl)-2-(2,4-dichlorophenyl)-5-methyl-1H-imidazol-4-yl)-1,3,4-thiadiazole

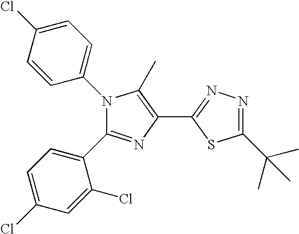

1-(4-Chlorophenyl)-2-(2,4-dichlorophenyl)-5-methyl-N′-pivaloyl-1H-imidazole-4-carbohydrazide (40 mg, 0.083 mmol) obtained in Step 1 of Example 1 was added to a microwave reactor containing Lawesson's reagent (101 mg, 0.25 mmol) in 1,4-dioxane (1 mL). The microwave reactor was capped and the mixture was heated at 180 □ for 30 min. The reaction mixture was then purified by preparative HPLC to provide the title compound (38 mg, 0.080 mmol, 95%) as a yellow solid.

¹H NMR (400 MHz, CDCl₃) δ 7.39-7.35 (m, 3H), 7.32 (d, J=1.8 Hz, 1H), 7.23 (dd, J=8.2, 2.3 Hz, 1H), 7.13-7.09 (m, 2H), 2.58 (s, 3H), 1.52 (s, 9H).

MH+477.

The following compounds of Examples 23 to 42 were obtained by repeating the procedure of Example 22.

### Example 23

### 2-(1-(4-Chlorophenyl)-2-(2,4-dichlorophenyl)-5-methyl-1H-imidazol-4-yl)-5-cyclohexyl-1,3,4-thiadiazole

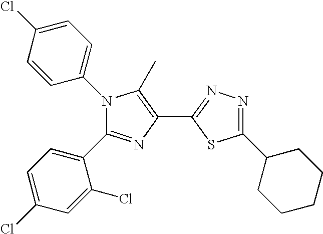

¹H NMR (400 MHz, CDCl₃) δ 7.38-7.32 (m, 4H), 7.24 (dd, J=8.2, 1.8 Hz, 1H), 7.11-7.07 (m, 2H), 3.20-3.14 (m, 1H), 2.58 (s, 3H), 2.21-2.17 (m, 2H), 1.89-1.84 (m, 2H), 1.77-1.74 (m, 1H), 1.65-1.56 (m, 1H), 1.50-1.40 (m, 2H), 1.36-1.25 (m, 2H).

MH+503.

### Example 24

### 2-(1-(4-Chlorophenyl)-2-(2,4-dichlorophenyl)-5-methyl-1H-imidazol-4-yl)-5-cyclopentyl-1,3,4-thiadiazole

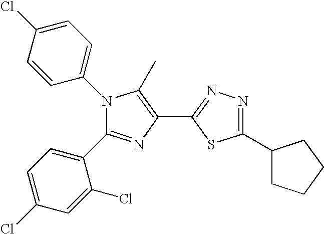

¹H NMR (400 MHz, CDCl₃) δ 7.38-7.32 (m, 4H), 7.24 (dd, J=8.2, 2.3 Hz, 1H), 7.11-7.07 (m, 2H), 3.63-3.55 (m, 1H), 2.58 (s, 3H), 2.28-2.22 (m, 2H), 1.94-1.81 (m, 4H), 1.75-1.71 (m, 2H).

MH+489.

### Example 25

### 2-(1-(4-Chlorophenyl)-2-(2,4-dichlorophenyl)-5-methyl-1H-imidazol-4-yl)-5-cyclobutyl-1,3,4-thiadiazole

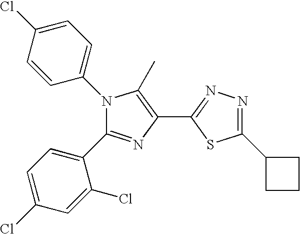

¹H NMR (400 MHz, CDCl₃) δ 7.39-7.33 (m, 4H), 7.24 (dd, J=8.7, 2.3 Hz, 1H), 7.11-7.08 (m, 2H), 4.03-3.97 (m, 1H), 2.59-2.51 (m, 5H), 2.47-2.38 (m, 2H), 2.15-2.01 (m, 2H).

MH+475.

### Example 26

### 2-(1-(4-Chlorophenyl)-2-(2,4-dichlorophenyl)-5-methyl-1H-imidazol-4-yl)-5-cycloheptyl-1,3,4-thiadiazole

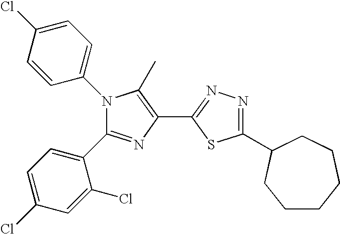

¹H NMR (400 MHz, CDCl₃) δ 7.38-7.32 (m, 4H), 7.24 (dd, J=8.7, 2.3 Hz, 1H), 7.11-7.07 (m, 2H), 3.43-3.36 (m, 1H), 2.58 (s, 3H), 2.23-2.16 (m, 2H), 1.89-1.80 (m, 4H), 1.70-1.59 (m, 6H).

MH+517.

### Example 27

### 2-(1-(4-Chlorophenyl)-2-(2,4-dichlorophenyl)-5-methyl-1H-imidazol-4-yl)-5-(hexan-2-yl)-1,3,4-thiadiazole

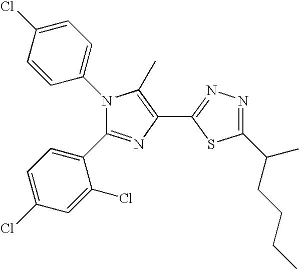

¹H NMR (400 MHz, CDCl₃) δ 7.38-7.33 (m, 4H), 7.24 (d, J=1.8 Hz, 1H), 7.11-7.08 (m, 2H), 3.41-3.32 (m, 1H), 2.59 (s, 3H), 1.85-1.68 (m, 2H), 1.44 (d, J=6.9 Hz, 3H), 1.38-1.28 (m, 4H), 0.88 (t, J=7.3 Hz, 3H).

MH+505.

### Example 28

### 2-(1-(4-Chlorophenyl)-2-(2,4-dichlorophenyl)-5-methyl-1H-imidazol-4-yl)-5-(pentan-2-yl)-1,3,4-thiadiazole

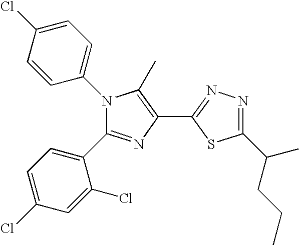

¹H NMR (400 MHz, CDCl₃) δ 7.39-7.33 (m, 4H), 7.24 (dd, J=8.2, 1.8 Hz, 1H), 7.11-7.08 (m, 2H), 3.43-3.34 (m, 1H), 2.59 (s, 3H), 1.84-1.66 (m, 2H), 1.45-1.33 (m, 5H), 0.92 (t, J=7.4 Hz, 3H).

MH+491.

### Example 29

### 2-sec-Butyl-5-(1-(4-chlorophenyl)-2-(2,4-dichlorophenyl)-5-methyl-1H-imidazol-4-yl)-1,3,4-thiadiazole

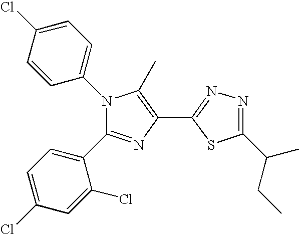

¹H NMR (400 MHz, CDCl₃) δ 7.39-7.33 (m, 4H), 7.24 (dd, J=8.2, 1.8 Hz, 1H), 7.11-7.08 (m, 2H), 3.32-3.25 (m, 1H), 2.59 (s, 3H), 1.88-1.74 (m, 2H), 1.44 (d, J=6.9 Hz, 3H), 0.97 (t, J=7.3 Hz, 3H).

MH+477.

### Example 30

### 2-(1-(4-Chlorophenyl)-2-(2,4-dichlorophenyl)-5-methyl-1H-imidazol-4-yl)-5-isopropyl-1,3,4-thiadiazole

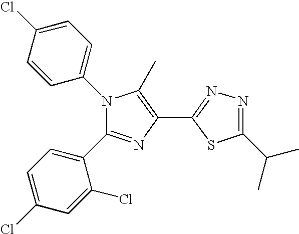

¹H NMR (400 MHz, CDCl₃) δ 7.39-7.35 (m, 3H), 7.33 (d, J=1.8 Hz, 1H), 7.24 (dd, J=8.2, 1.8 Hz, 1H), 7.11-7.08 (m, 2H), 3.52-3.45 (m, 1H), 2.58 (s, 3H), 1.47 (d, J=6.9 Hz, 6H).

MH+463.

### Example 31

### 2-(1-(4-Chlorophenyl)-2-(2,4-dichlorophenyl)-5-methyl-1H-imidazol-4-yl)-5-(pentan-3-yl)-1,3,4-thiadiazole

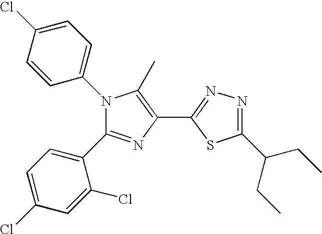

¹H NMR (400 MHz, CDCl₃) δ 7.38-7.32 (m, 4H), 7.27-7.23 (m, 1H), 7.11-7.08 (m, 2H), 3.11-3.09 (m, 1H), 2.60 (s, 3H), 1.91-1.68 (m, 4H), 0.93 (t, J=7.2 Hz, 6H).

MH+491.

### Example 32

### 2-(1-(4-Chlorophenyl)-2-(2,4-dichlorophenyl)-5-methyl-1H-imidazol-4-yl)-5-(1-phenylethyl)-1,3,4-thiadiazole

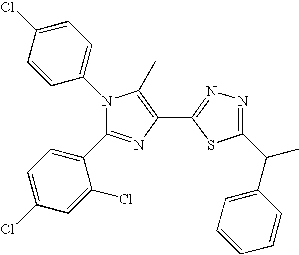

¹H NMR (400 MHz, CDCl₃) δ 7.37-7.33 (m, 5H), 7.31-7.28 (m, 4H), 7.22 (dd, J=8.2, 1.8 Hz, 1H), 7.09-7.05 (m, 2H), 4.62 (q, J=7.4 Hz, 1H), 2.58 (s, 3H), 1.87 (d, J=7.4 Hz, 3H).

MH+525.

### Example 33

### 2-(1-(4-Chlorophenyl)-2-(2,4-dichlorophenyl)-5-ethyl-1H-imidazol-4-yl)-5-cyclohexyl-1,3,4-thiadiazole

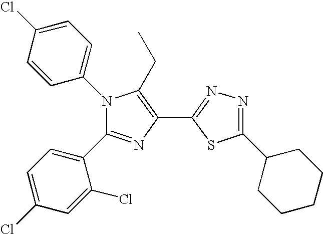

¹H NMR (400 MHz, CDCl₃) δ 7.39-7.35 (m, 2H), 7.33-7.31 (m, 2H), 7.23 (dd, J=8.2, 2.3 Hz, 1H), 7.16-7.12 (m, 2H), 3.20-3.12 (m, 1H), 3.05 (q, J=7.4 Hz, 2H), 2.15 (d, J=11 Hz, 2H), 1.89-1.84 (m, 2H), 1.78-1.73 (m, 1H), 1.66-1.56 (m, 1H), 1.51-1.40 (m, 2H), 1.36-1.25 (m, 2H), 1.12 (t, J=7.3 Hz, 3H).

MH+517.

### Example 34

### 2-(1-(4-Chlorophenyl)-2-(2,4-dichlorophenyl)-5-ethyl-1H-imidazol-4-yl)-5-cyclopentyl-1,3,4-thiadiazole

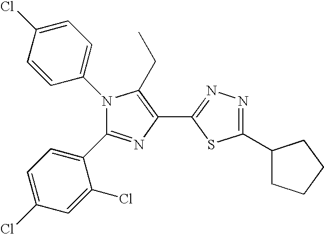

¹H NMR (400 MHz, CDCl₃) δ 7.39-7.35 (m, 2H), 7.33-7.31 (m, 2H), 7.23 (dd, J=8.2, 1.8 Hz, 1H), 7.15-7.12 (m, 2H), 3.62-3.55 (m, 1H), 3.05 (q, J=7.4 Hz, 2H), 2.28-2.22 (m, 2H), 1.95-1.81 (m, 4H), 1.75-1.72 (m, 2H), 1.12 (t, J=7.4 Hz, 3H).

MH+503.

### Example 35

### 2-(1-(4-Chlorophenyl)-2-(2,4-dichlorophenyl)-5-ethyl-1H-imidazol-4-yl)-5-cyclobutyl-1,3,4-thiadiazole

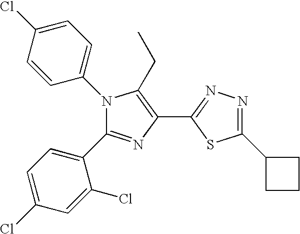

¹H NMR (400 MHz, CDCl₃) δ 7.38-7.35 (m, 2H), 7.34-7.32 (m, 2H), 7.23 (dd, J=8.2, 1.8 Hz, 1H), 7.16-7.12 (m, 2H), 4.03-3.95 (m, 1H), 3.05 (q, J=7.3 Hz, 2H), 2.59-2.51 (m, 2H), 2.48-2.38 (m, 2H), 2.15-2.03 (m, 2H), 1.12 (t, J=7.3 Hz, 3H).

MH+489.

### Example 36

### 2-tert-Butyl-5-(1-(4-chlorophenyl)-2-(2,4-dichlorophenyl)-5-ethyl-1H-imidazol-4-yl)-1,3,4-thiadiazole

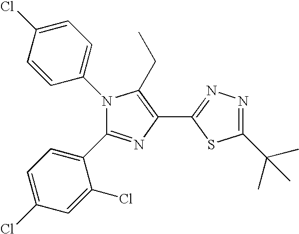

¹H NMR (300 MHz, CDCl₃) δ 7.39-7.31 (m, 4H), 7.23 (dd, J=8.2, 1.8 Hz, 1H), 7.15-7.13 (m, 2H), 3.05 (q, J=7.3 Hz, 2H), 1.52 (s, 9H), 1.13 (t, J=7.3 Hz, 3H).

MH+491.

### Example 37

### 2-(1-(4-Chlorophenyl)-2-(2,4-dichlorophenyl)-5-ethyl-1H-imidazol-4-yl)-5-(hexan-2-yl)-1,3,4-thiadiazole

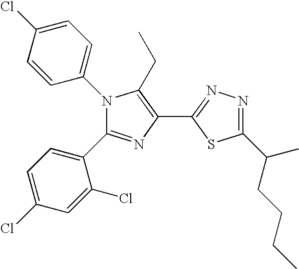

¹H NMR (300 MHz, CDCl₃) δ 7.39-7.31 (m, 4H), 7.23 (dd, J=8.2, 2.0 Hz, 1H), 7.16-7.12 (m, 2H), 3.39-3.32 (m, 1H), 3.06 (q, J=7.3 Hz, 2H), 1.83-1.64 (m, 2H), 1.44 (d, J=7.0 Hz, 3H), 1.35-1.32 (m, 4H), 1.12 (t, J=7.3 Hz, 3H), 0.88 (t, J=6.8 Hz, 3H).

MH+519.

### Example 38

### 2-(1-(4-Chlorophenyl)-2-(2,4-dichlorophenyl)-5-ethyl-1H-imidazol-4-yl)-5-(pentan-2-yl)-1,3,4-thiadiazole

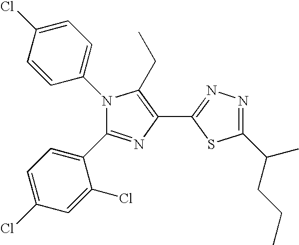

¹H NMR (400 MHz, CDCl₃) δ 7.38-7.31 (m, 4H), 7.26-7.21 (m, 1H), 7.15-7.12 (m, 2H), 3.41-3.36 (m, 1H), 3.05 (q, J=7.6 Hz, 2H), 1.83-1.65 (m, 2H), 1.44-1.35 (m, 5H), 1.12 (t, J=7.6 Hz, 3H), 0.93 (t, J=7.2 Hz, 3H).

MH+505.

### Example 39

### 2-sec-Butyl-5-(1-(4-chlorophenyl)-2-(2,4-dichlorophenyl)-5-ethyl-1H-imidazol-4-yl)-1,3,4-thiadiazole

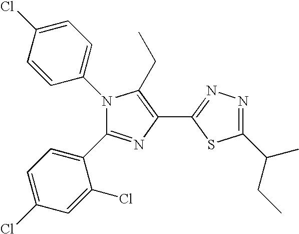

¹H NMR (400 MHz, CDCl₃) δ 7.39-7.32 (m, 4H), 7.25-7.22 (m, 1H), 7.16-7.13 (m, 2H), 3.32-3.27 (m, 1H), 3.06 (q, J=7.6 Hz, 2H), 1.89-1.74 (m, 2H), 1.44 (d, J=6.8 Hz, 3H), 1.12 (t, J=7.2 Hz, 3H), 0.98 (t, J=7.6 Hz, 3H).

MH+491

### Example 40

### 2-(1-(4-Bromophenyl)-2-(2,4-dichlorophenyl)-5-methyl-1H-imidazol-4-yl)-5-cyclohexyl-1,3,4-thiadiazole

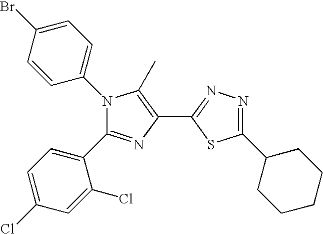

¹H NMR (400 MHz, CDCl₃) δ 7.52 (d, J=8.0 Hz, 2H), 7.35-7.33 (m, 2H), 7.26-7.24 (m, 1H), 7.03 (d, J=8.0 Hz, 2H), 3.21-3.14 (m, 1H), 2.59 (s, 3H), 2.21-2.18 (m, 2H), 1.89-1.85 (m, 2H), 1.77-1.74 (m, 1H), 1.65-1.55 (m, 2H), 1.50-1.40 (m, 2H), 1.36-1.25 (m, 1H).

MH+547.

### Example 41

### 2-(1-(4-Bromophenyl)-2-(2,4-dichlorophenyl)-5-methyl-1H-imidazol-4-yl)-5-tert-butyl-1,3,4-thiadiazole

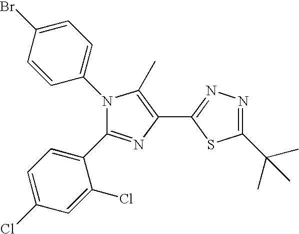

¹H NMR (400 MHz, CDCl₃) δ 7.54-7.51 (m, 2H), 7.35-7.33 (m, 2H), 7.25 (dd, J=8.2, 2.0 Hz, 1H), 7.06-7.02 (m, 2H), 2.59(s, 3H), 1.53 (s, 9H).

MH+521.

### Example 42

### 2-(1-(4-Bromophenyl)-2-(2,4-dichlorophenyl)-5-ethyl-1H-imidazol-4-yl)-5-tert-butyl-1,3,4-thiadiazole

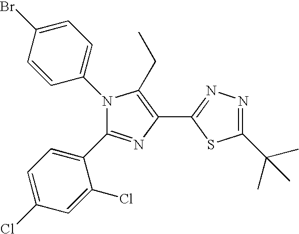

¹H NMR (400 MHz, CDCl₃) δ 7.54-7.51 (m, 2H), 7.34-7.32 (m, 2H), 7.23 (dd, J=8.4, 2.0 Hz, 1H), 7.09-7.06 (m, 2H), 3.04 (q, J=7.6 Hz, 2H), 1.52 (s, 9H), 1.13 (t, J=7.6 Hz, 3H).

MH+535.

### Preparation of oxazole (Formula (1c))

### Example 43

### 2-(1-(4-Chlorophenyl)-2-(2,4-dichlorophenyl)-5-methyl-1H-imidazol-4-yl)-5-cyclohexyloxazole

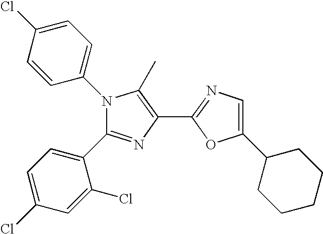

Step 1: tert-Butyl 2-(methoxy(methyl)amino)-2-oxoethylcarbamate

2-(tert-Butoxycarbonylamino)acetic acid (800 mg, 4.57 mmol), N,O-dimethylhydroxylamine hydrochloride (535 mg, 5.48 mmol), EDCI (1.05 g, 5.48 mmol) and HOBt (740 mg, 5.48 mmol) were dissolved in DCM (25 mL), to which NMM (2.77 g, 27.42 mmol) was added at room temperature. The reaction mixture was stirred overnight. After the reaction was completed, DCM was evaporated in vacuo. The residue was dissolved in MeOH, filtered through a syringe filter, and then purified by reverse-phase preparative HPLC to provide the desired product (705 mg, 71%) as a white solid

¹H NMR (400 MHz, CDCl₃) δ 5.27 (br, 1H), 4.09 (d, J=4.6 Hz, 2H), 3.72 (s, 3H), 3.21 (s, 3H) 1.46 (s, 9H).

MH+219.

Step 2: tert-Butyl 2-cyclohexyl-2-oxoethylcarbamate

tert-Butyl 2-(methoxy(methyl)amino)-2-oxoethylcarbamate (400 mg, 1.83 mmol) obtained in Step 1 was dissolved in THF (10 mL), to which cyclohexylmagnesium chloride (4.12 mL, 8.24 mmol) was added portionwise at room temperature under N₂ atmosphere. As addition of the Grignard reagent was completed, the reaction mixture was stirred and heated to reflux for an hour. Then, the mixture was allowed to cool down to room temperature. The reaction was quenched by adding water (10 mL) slowly. 1N HCl solution (30 mL) was added to the mixture, and extracted with EtOAc (20 mL×3). The combined organic layers were collected and evaporated in vacuo. The crude material was purified by column chromatography (hexane: EtOAc=10:1 to 5:1) to yield the desired product (110 mg, 25%).

¹H NMR (400 MHz, CDCl₃) δ 5.26 (br, 1H), 4.07 (d, J=4.2 Hz, 2H), 2.41-2.34 (m, 1H), 1.86-1.67 (m, 5H), 1.45 (s, 9H), 1.43-1.19 (m, 5H).

MH+242.

### Step 3:

### 1-(4-Chlorophenyl)-N-(2-cyclohexyl-2-oxoethyl)-2-(2,4-dichlorophenyl)-5-methyl-1H-imidazole-4-carboxamide

tert-Butyl 2-cyclohexyl-2-oxoethylcarbamate (85 mg, 0.423 mmol) obtained in Step 2 was dissolved in DCM (2 mL), to which TFA (1 mL) was added at room temperature. The reaction was continued for an hour at room temperature. The volatiles were removed under vacuum. The residue (2-amino-1-cyclohexylethanone trifluoroacetic acid), 1-(4-chlorophenyl)-2-(2,4-dichlorophenyl)-5-methyl-1H-imidazole-4-carboxylic acid (161 mg, 0.423 mmol), EDCI (81.1 mg, 0.423 mmol), and HOBt (57.2 mmol, 0.423 mmol) in DCM (3 mL) was added NMM (0.43 g, 4.23 mmol) at room temperature. The reaction mixture was stirred overnight. DCM was removed by evaporation under vacuum. The residue was purified by reverse-phase preparative HPLC to afford the title compound (29 mg, 16%).

¹H NMR (300 MHz, CDCl₃) δ 7.76 (br, 1H), 7.39-7.31 (m, 3H), 7.26-7.21 (m, 2H), 7.03 (d, J=8.6 Hz, 2H), 4.34 (d, J=5.1 Hz), 2.52-2.42 (m, 4H), 1.91-1.67 (m, 5H), 1.49-1.16 (m, 5H).

MH+504.

### Step 4:

### 2-(1-(4-Chlorophenyl)-2-(2,4-dichlorophenyl)-5-methyl-1H-imidazol-4-yl)-5-cyclohexyloxazole

1-(4-Chlorophenyl)-N-(2-cyclohexyl-2-oxoethyl)-2-(2,4-dichlorophenyl)-5-methyl-1H-imidazole-4-carboxamide (29 mg, 0.0574 mmol) obtained in Step 3 was added to a microwave reactor containing Burgess reagent (27.4 mg, 0.115 mmol) in THF (1 mL). The microwave reactor was capped and the mixture was irradiated at 160 for 30 min. The reaction product was purified by reverse-phase preparative HPLC to provide the title compound (6.2 mg, 22%) as a white solid.

¹H NMR (400 MHz, CDCl₃) δ 7.39-7.30 (m, 4H), 7.25-7.22 (m, 1H), 7.09-7.05 (m, 2H), 6.80 (s, 1H), 2.79-2.75 (m, 1H), 2.49 (s, 3H), 2.11 (d, J=12.4 Hz, 2H), 1.82-1.79 (m, 2H), 1.70-1.65 (m, 2H), 1.49-1.25 (m, 4H).

MH+486

The following compounds of Examples 44 to 51 were obtained by repeating the procedure of Example 43.

### Example 44

### 2-(1-(4-Chlorophenyl)-2-(2,4-dichlorophenyl)-5-methyl-1H-imidazol-4-yl)-5-ethyloxazole

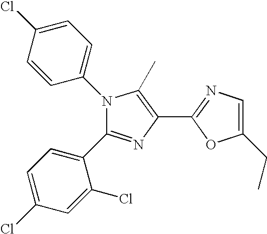

¹H NMR (300 MHz, CDCl₃) δ 7.40-7.35 (m, 3H), 7.31 (d, J=2.0 Hz, 1H), 7.24 (dd, J=8.2, 2.2 Hz, 1H), 7.10-7.07 (m, 2H), 6.84 (s, 1H), 2.77 (q, J=7.5 Hz, 2H), 2.51 (s, 3H), 1.30 (t, J=7.5 Hz, 3H).

MH+432.

### Example 45

### 5-Butyl-2-(1-(4-chlorophenyl)-2-(2,4-dichlorophenyl)-5-methyl-1H-imidazol-4-yl)oxazole

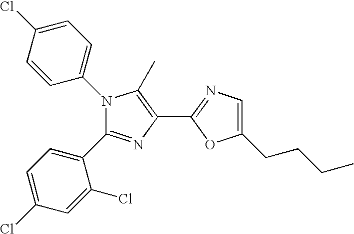

¹H NMR (300 MHz, CDCl₃) δ 7.40-7.22 (m, 5H), 7.10-7.07 (m, 2H), 6.84 (s, 1H), 2.73 (t, J=7.5 Hz, 2H), 2.51 (s, 3H), 1.72-1.64 (m, 2H), 1.44-1.37 (m, 2H), 0.94 (t, J=7.3 Hz, 3H).

MH+460.

### Example 46

### 2-(1-(4-Chlorophenyl)-2-(2,4-dichlorophenyl)-5-methyl-1H-imidazol-4-yl)-5-isopropyloxazole

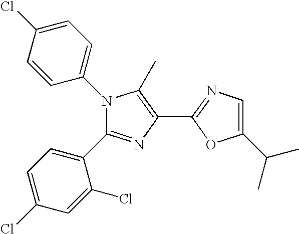

¹H NMR (300 MHz, CDCl₃) δ 7.41-7.34 (m, 3H), 7.30 (d, J=2.0 Hz, 1H), 7.24 (dd, J=8.2, 2.0 Hz, 1H), 7.09-7.07 (m, 2H), 6.81 (s, 1H), 3.11-3.06 (m, 1H), 2.50 (s, 3H), 1.32 (d, J=7.0 Hz, 6H).

MH+446.

### Example 47

### 2-(1-(4-Chlorophenyl)-2-(2,4-dichlorophenyl)-5-methyl-1H-imidazol-4-yl)-5-isobutyloxazole

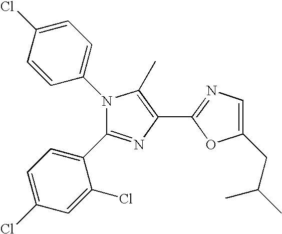

¹H NMR (400 MHz, CDCl₃) δ 7.40-7.22 (m, 5H), 7.09-7.07 (m, 2H), 6.85 (s, 1H), 2.60 (d, J=6.8 Hz, 2H), 2.51 (s, 3H), 2.08-2.03 (m, 1H), 0.97 (d, J=6.6 Hz, 6H).

MH+460.

### Example 48

### 2-tert-Butyl-5-(1-(4-chlorophenyl)-2-(2,4-dichlorophenyl)-5-methyl-1H-imidazol-4-yl)oxazole

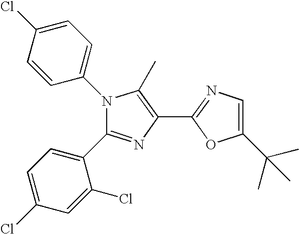

¹H NMR (400 MHz, CDCl₃) δ 7.41-7.33 (m, 3H), 7.30 (d, J=1.8 Hz, 1H), 7.24 (dd, J=8.2, 1.8 Hz, 1H), 7.09-7.05 (m, 2H), 6.79 (s, 1H), 2.49 (s, 3H), 1.36 (s, 9H).

MH+460.

### Example 49

### 2-tert-Butyl-5-(1-(4-chlorophenyl)-2-(2,4-dichlorophenyl)-5-ethyl-1H-imidazol-4-yl)oxazole

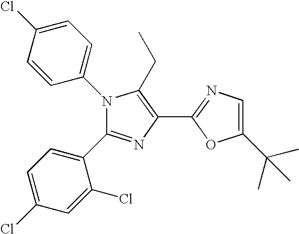

¹H NMR (400 MHz, CDCl₃) δ 7.39-7.34 (m, 3H), 7.29 (d, J=2.3 Hz, 1H), 7.22 (dd, J=8.2, 2.3 Hz, 1H), 7.13-7.10 (m, 2H), 6.80 (s, 1H), 2.95 (q, J=7.3 Hz, 2H), 1.36 (s, 9H), 1.09 (t, J=7.3 Hz, 3H).

MH+474.

### Example 50

### 2-(1-(4-Bromophenyl)-2-(2,4-dichlorophenyl)-5-methyl-1H-imidazol-4-yl)-5-tert-butyloxazole

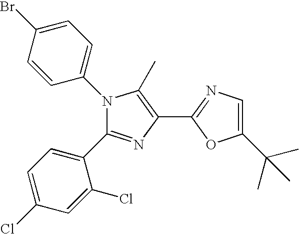

¹H NMR (400 MHz, CDCl₃) δ 7.53-7.50 (m, 2H), 7.42-7.40 (m, 1H), 7.31-7.30 (m, 1H), 7.27-7.23 (m, 1H), 7.02-7.00 (m, 2H), 6.80 (s, 1H), 2.49 (s, 3H), 1.37 (s, 9H).

MH+504.

### Example 51

### 2-(1-(4-Bromophenyl)-2-(2,4-dichlorophenyl)-5-ethyl-1H-imidazol-4-yl)-5-tert-butyloxazole

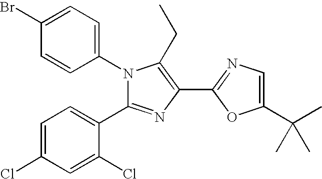

¹H NMR (400 MHz, CDCl₃) δ 7.52-7.50 (m, 2H), 7.38 (d, J=8.4 Hz, 1H), 7.30 (d, J=2.0 Hz, 1H), 7.22 (dd, J=8.4, 2.0 Hz, 1H), 7.06-7.04 (m, 2H), 6.80 (s, 1H), 2.95 (q, J=7.2 Hz, 2H), 1.36 (s, 9H), 1.09 (t, J=7.6 Hz, 3H).

MH+518.

### Preparation of thiazole (Formula (Id))

### Example 52

### 2-(1-(4-chlorophenyl)-2-(2,4-dichlorophenyl)-5-methyl-1H-imidazol-4-yl)-5-cyclohexylthiazole

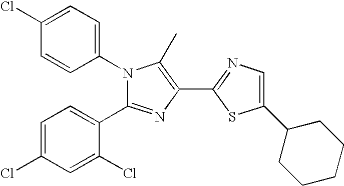

1-(4-Chlorophenyl)-N-(2-cyclohexyl-2-oxoethyl)-2-(2,4-dichlorophenyl)-5-methyl-1H-imidazole-4-carboxamide (35 mg, 0.0693 mmol) obtained in Step 3 of Example 43 was added to a microwave reactor containing Lawesson's reagent (0.18 g, 0.44 mmol) in THF (1 mL). The microwave reactor was capped and the mixture was irradiated at 160 for 30 min. The reaction product was purified by reverse-phase preparative HPLC to provide the title compound (11.3 mg, 32%) as a white solid.

¹H NMR (300 MHz, CDCl₃) δ 7.50 (s, 1H), 7.38-7.33 (m, 4H), 7.23 (dd, J=8.2, 2.0 Hz, 1H), 7.10-7.06 (m, 2H), 2.89-2.83 (m, 1H), 2.53 (s, 3H), 2.09-2.05 (m, 2H), 1.85-1.71 (m, 2H), 1.53-1.25 (m, 6H).

MH+502.

The following compounds of Examples 53 to 60 were obtained by repeating the procedure of Example 52.

### Example 53

### 2-(1-(4-Chlorophenyl)-2-(2,4-dichlorophenyl)-5-methyl-1H-imidazol-4-yl)-5-ethylthiazole

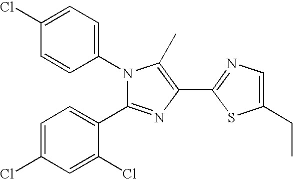

¹H NMR (300 MHz, CDCl₃) δ 7.49 (s, 1H) 7.38-7.34 (m, 3H), 7.31 (d, J=1.8 Hz, 1H), 7.23 (dd, J=8.2, 2.0 Hz, 1H), 7.10-7.02 (m, 2H), 2.89 (q, J=7.5 Hz, 2H), 2.53 (s, 3H), 1.34 (t, J=7.5 Hz, 3H).

MH+448.

### Example 54

### 5-Butyl-2-(1-(4-chlorophenyl)-2-(2,4-dichlorophenyl)-5-methyl-1H-imidazol-4-yl)thiazole

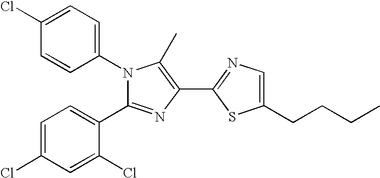

¹H NMR (300 MHz, CDCl₃) δ 7.48 (s, 1H) 7.38-7.33 (m, 3H), 7.31 (d, J=1.8 Hz, 1H), 7.24 (dd, J=8.2, 2.0 Hz, 1H), 7.10-7.07 (m, 2H), 2.85 (t, J=7.0 Hz, 2H), 2.53 (s, 3H), 1.73-1.62 (m, 2H), 1.47-1.34 (m, 2H), 0.93 (t, J=7.3 Hz, 3H).

MH+476.

### Example 55

### 2-(1-(4-Chlorophenyl)-2-(2,4-dichlorophenyl)-5-methyl-1H-imidazol-4-yl)-5-isopropylthiazole

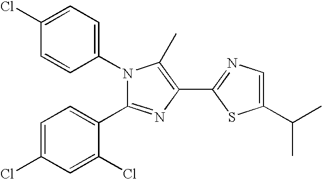

¹H NMR (300 MHz, CDCl₃) δ 7.52 (s, 1H), 7.39-7.34 (m, 3H), 7.31 (d, J=2.0 Hz, 1H), 7.23 (dd, J=8.2, 2.0 Hz, 1H), 7.10-7.07 (m, 2H), 3.27-3.18 (m, 1H), 2.52 (s, 3H), 1.36 (d, J=6.8 Hz, 6H).

MH+462.

### Example 56

### 2-(1-(4-Chlorophenyl)-2-(2,4-dichlorophenyl)-5-methyl-1H-imidazol-4-yl)-5-isobutylthiazole

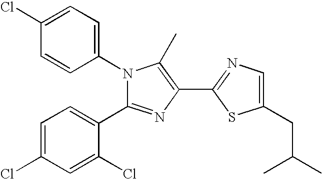

¹H NMR (400 MHz, CDCl₃) δ 7.47 (s, 1H), 7.38-7.22 (m, 5H), 7.08 (d, J=8.0 Hz, 2H) 2.71 (d, J=7.2 Hz, 2H), 2.53 (s, 3H), 1.93-1.86 (m, 1H), 0.97 (d, J=6.4 Hz, 6H).

MH+476.

### Example 57

### 2-tert-Butyl-5-(1-(4-chlorophenyl)-2-(2,4-dichlorophenyl)-5-methyl-1H-imidazol-4-yl)thiazole

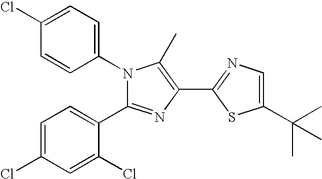

¹H NMR (400 MHz, CDCl₃) δ 7.52 (s, 1H), 7.37-7.34 (m, 3H), 7.31 (d, J=1.8 Hz, 1H), 7.23 (dd, J=8.2, 1.8 Hz, 1H), 7.08 (d, J=8.2 Hz, 2H), 2.52 (s, 3H), 1.42 (s, 9H).

MH+476.

### Example 58

### 2-tert-Butyl-5-(1-(4-chlorophenyl)-2-(2,4-dichlorophenyl)-5-ethyl-1H-imidazol-4-yl)thiazole

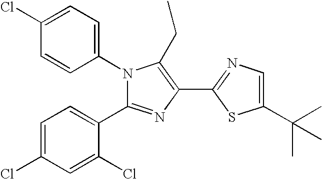

¹H NMR (400 MHz, CDCl₃) δ 7.51 (s, 1H), 7.37-7.33 (m, 3H), 7.30 (d, J=2.3 Hz, 1H), 7.21 (dd, J=8.2, 1.8 Hz, 1H), 7.14-7.11 (m, 2H), 3.01 (q, J=7.3 Hz, 2H), 1.42 (s, 9H), 1.07 (t, J=7.3 Hz, 3H).

MH+490.

### Example 59

### 2-(1-(4-Bromophenyl)-2-(2,4-dichlorophenyl)-5-methyl-1H-imidazol-4-yl)-5-tert-butylthiazole

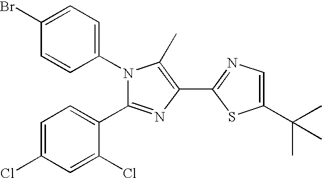

¹H NMR (400 MHz, CDCl₃) δ 7.53-7.49 (m, 3H), 7.38-7.36 (m, 1H), 7.31 (d, J=1.8 Hz, 1H), 7.23 (dd, J=8.2, 1.8 Hz, 1H), 7.04-7.00 (m, 2H), 2.53 (s, 3H), 1.42 (s, 9H).

MH+520.

### Example 60

### 2-(1-(4-Bromophenyl)-2-(2,4-dichlorophenyl)-5-ethyl-1H-imidazol-4-yl)-5-tert-butylthiazole

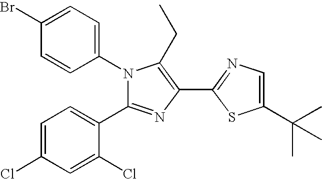

¹H NMR (400 MHz, CDCl₃) δ 7.52-7.50 (m, 3H), 7.36-7.34 (m, 2H), 7.31 (d, J=1.6 Hz, 1H), 7.22 (dd, J=8.4, 2.0 Hz, 1H), 7.08-7.05 (m, 2H), 3.02 (q, J=7.6 Hz, 2H), 1.42 (s, 9H), 1.07 (t, J=7.6 Hz, 3H).

MH+534.

### Pharmacological Test: In Vitro Activity Analysis

The compounds of the present invention were analyzed for their binding characteristics for CB₁ and CB₂ and the pharmacological activity thereof in accordance with the method disclosed in [Devane W A, Dysarz F A 3^{rd}, Johnson M R, Melvin L S and Howlett A C, Determination and characterization of a cannabinoid receptor in rat brain, Mol. Pharmacol., 34(5): 605-13(1998)]. The analysis was performed using [³H]CP-55940 which is a selectively radioactivity-labeled 5-(1,1-dimethyheptyl)-2[5-hydroxy-2-(3-hydroxypropyl)-cyclohexyl]-phenol, purchased from PerkinElmer Life Sciences, Inc. (Boston, Mass., U.S.A.), through a rat CB-1 receptor binding protocol as follows.

The tissue obtained from the brain of SD rats was homogenized with a Dounce homogenate system in TME (50 mM Tris, 3 mM MgCl₂ and 1 mM EDTA, pH 7.4) at 4° C., and the homogenate was centrifuged at 48,000 g for 30 min. at 4° C. The pellet was resuspended in 5 ml of TME and the suspension was divided into aliquots and stored at −70° C. until its use in the following assay.

2 μl of the test compound was diluted in dimethylsulphoxide and was added to a deep well of a polypropylene plate, to which 50 μl of [³H]CP-55940 diluted in a ligand buffer solution (0.1% bovine serum albumin(BSA)+TME) was added. The tissue concentrations were determined by Bradford protein analysis, and 148 μl of brain tissue of the required concentration was added to the plate. The plate was covered and placed in a 30° C. incubator for 60 min, and then transformed on GF/B filtermat pretreated in polyethylenimine (PEI) using a cell harvester. Each filter was washed five times and dried at 60° C. for 1 hr. Then, the degree of radioactivity retained by the filter was measured using Wallac Microbeta™ (PerkinElmer Life Sciences, Inc., Massachusetts, U.S.A.) and the activity of the compound for inhibiting CB₁ receptor was determined therefrom and compared with that of the control which is known as a cannabinoid CB₁ receptor antagonist in the Annual Reports in Medicinal Chemistry, Volume 40, 2005, pp 104-105. The results are shown in Table 1.

TABLE 1

| | | | IC₅₀ |
|---|---|---|---|
| Example | | Compound | (nM) |
| 1 | 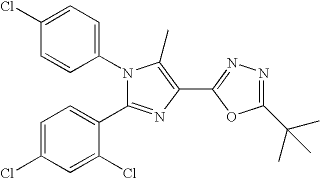 | 2-tert-Butyl-5-(1-(4-chlorophenyl)-2-(2,4-dichlorophenyl)-5-methyl-1H-imidazol-4-yl)-1,3,4-oxadiazole | 13.2 |
| 2 | 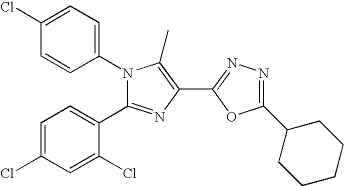 | 2-(1-(4-Chlorophenyl)-2-(2,4-dichlorophenyl)-5-methyl-1H-imidazol-4-yl)-5-cyclohexyl-1,3,4-oxadiazole | 115 |
| 3 | 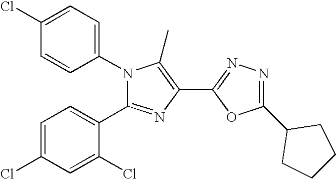 | 2-(1-(4-Chlorophenyl)-2-(2,4-dichlorophenyl)-5-methyl-1H-imidazol-4-yl)-5-cyclopentyl-1,3,4-oxadiazole | 35.3 |
| 4 | 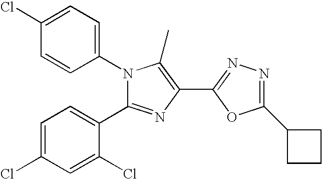 | 2-(1-(4-Chlorophenyl)-2-(2,4-dichlorophenyl)-5-methyl-1H-imidazol-4-yl)-5-cyclobutyl-1,3,4-oxadiazole | 38.7 |
| 5 | 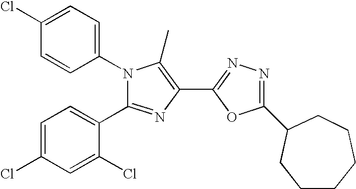 | 2-(1-(4-Chlorophenyl)-2-(2,4-dichlorophenyl)-5-methyl-1H-imidazol-4-yl)-5-cycloheptyl-1,3,4-oxadiazole | 27.6 |
| 6 | 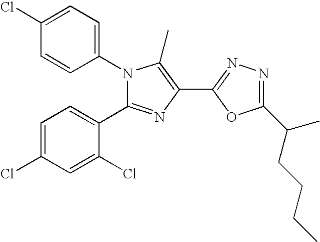 | 2-(1-(4-Chlorophenyl)-2-(2,4-dichlorophenyl)-5-methyl-1H-imidazol-4-yl)-5-(hexan-2-yl)-1,3,4-oxadiazole | 12.9 |
| 7 | 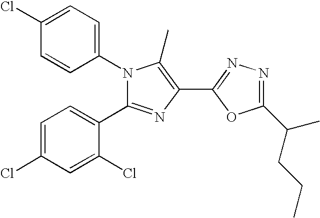 | 2-(1-(4-Chlorophenyl)-2-(2,4-dichlorophenyl)-5-methyl-1H-imidazol-4-yl)-5-(pentan-2-yl)-1,3,4-oxadiazole | 14.2 |
| 8 | 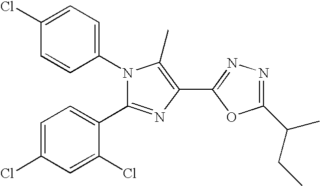 | 2-sec-Butyl-5-(1-(4-chlorophenyl)-2-(2,4-dichlorophenyl)-5-methyl-1H-imidazol-4-yl)-1,3,4-oxadiazole | 39.4 |
| 9 | 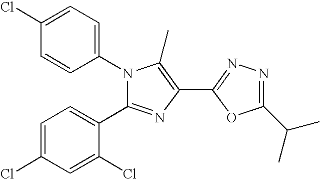 | 2-(1-(4-Chlorophenyl)-2-(2,4-dichlorophenyl)-5-methyl-1H-imidazol-4-yl)-5-isopropyl-1,3,4-oxadiazole | 25.8 |
| 10 | 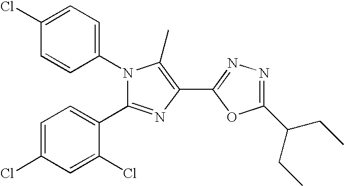 | 2-(1-(4-Chlorophenyl)-2-(2,4-dichlorophenyl)-5-methyl-1H-imidazol-4-yl)-5-(pentan-3-yl)-1,3,4-oxadiazole | 22.3 |
| 11 | 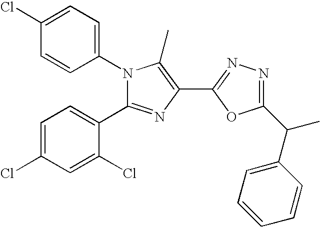 | 2-(1-(4-Chlorophenyl)-2-(2,4-dichlorophenyl)-5-methyl-1H-imidazol-4-yl)-5-(1-phenylethyl)-1,3,4-oxadiazole | 31.4 |
| 12 | 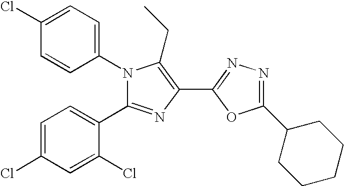 | 2-(1-(4-Chlorophenyl)-2-(2,4-dichlorophenyl)-5-ethyl-1H-imidazol-4-yl)-5-cyclohexyl-1,3,4-oxadiazole | 48.4 |
| 13 | 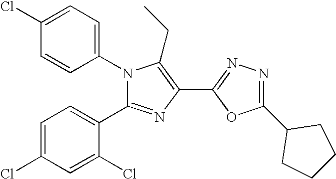 | 2-(1-(4-Chlorophenyl)-2-(2,4-dichlorophenyl)-5-ethyl-1H-imidazol-4-yl)-5-cyclopentyl-1,3,4-oxadiazole | 25.8 |
| 14 | 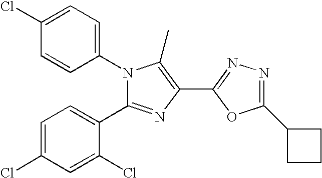 | 2-(1-(4-Chlorophenyl)-2-(2,4-dichlorophenyl)-5-ethyl-1H-imidazol-4-yl)-5-cyclobutyl-1,3,4-oxadiazole | 34.1 |
| 15 | 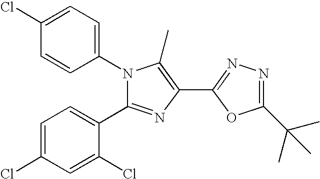 | 2-tert-Butyl-5-(1-(4-chlorophenyl)-2-(2,4-dichlorophenyl)-5-ethyl-1H-imidazol-4-yl)-1,3,4-oxadiazole | 18.1 |
| 16 | 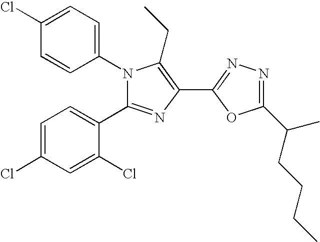 | 2-(1-(4-Chlorophenyl)-2-(2,4-dichlorophenyl)-5-ethyl-1H-imidazol-4-yl)-5-(hexan-2-yl)-1,3,4-oxadiazole | 11.2 |
| 17 | 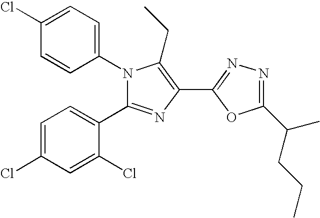 | 2-(1-(4-Chlorophenyl)-2-(2,4-dichlorophenyl)-5-ethyl-1H-imidazol-4-yl)-5-(pentan-2-yl)-1,3,4-oxadiazole | 11.5 |
| 18 | 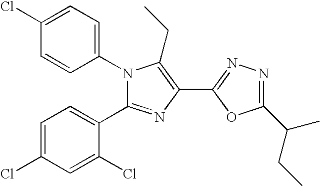 | 2-sec-Butyl-5-(1-(4-chlorophenyl)-2-(2,4-dichlorophenyl)-5-ethyl-1H-imidazol-4-yl)-1,3,4-oxadiazole | 17.9 |
| 19 | 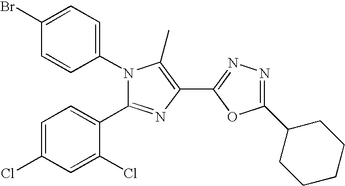 | 2-(1-(4-Bromophenyl)-2-(2,4-dichlorophenyl)-5-methyl-1H-imidazol-4-yl)-5-cyclohexyl-1,3,4-oxadiazole | 70.0 |
| 20 | 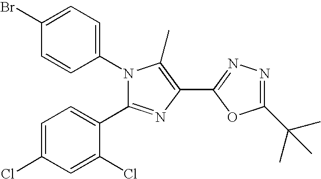 | 2-(1-(4-Bromophenyl)-2-(2,4-dichlorophenyl)-5-methyl-1H-imidazol-4-yl)-5-tert-butyl-1,3,4-oxadiazole | 5.81 |
| 21 | 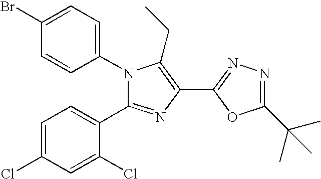 | 2-(1-(4-Bromophenyl)-2-(2,4-dichlorophenyl)-5-ethyl-1H-imidazol-4-yl)-5-tert-butyl-1,3,4-oxadiazole | 3.15 |
| 22 | 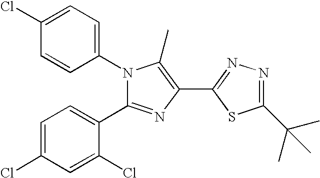 | 2-tert-Butyl-5-(1-(4-chlorophenyl)-2-(2,4-dichlorophenyl)-5-methyl-1H-imidazol-4-yl)-1,3,4-thiadiazole | 20.2 |
| 23 | 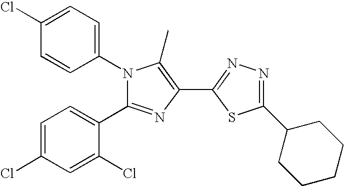 | 2-(1-(4-Chlorophenyl)-2-(2,4-dichlorophenyl)-5-methyl-1H-imidazol-4-yl)-5-cyclohexyl-1,3,4-thiadiazole | 46.0 |
| 24 | 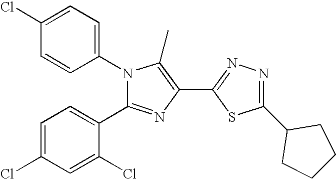 | 2-(1-(4-Chlorophenyl)-2-(2,4-dichlorophenyl)-5-methyl-1H-imidazol-4-yl)-5-cyclopentyl-1,3,4-thiadiazole | 10.8 |
| 25 | 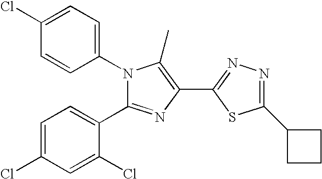 | 2-(1-(4-Chlorophenyl)-2-(2,4-dichlorophenyl)-5-methyl-1H-imidazol-4-yl)-5-cyclobutyl-1,3,4-thiadiazole | 42.3 |
| 26 | 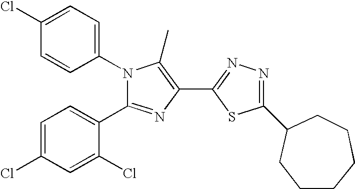 | 2-(1-(4-Chlorophenyl)-2-(2,4-dichlorophenyl)-5-methyl-1H-imidazol-4-yl)-5-cycloheptyl-1,3,4-thiadiazole | 21.6 |
| 27 | 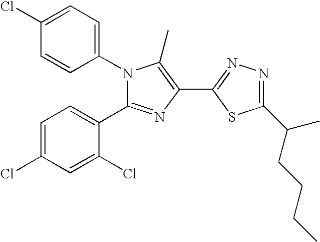 | 2-(1-(4-Chlorophenyl)-2-(2,4-dichlorophenyl)-5-methyl-1H-imidazol-4-yl)-5-(hexan-2-yl)-1,3,4-thiadiazole | 15.0 |
| 28 | 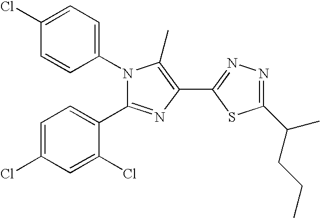 | 2-(1-(4-Chlorophenyl)-2-(2,4-dichlorophenyl)-5-methyl-1H-imidazol-4-yl)-5-(pentan-2-yl)-1,3,4-thiadiazole | 12.7 |
| 29 | 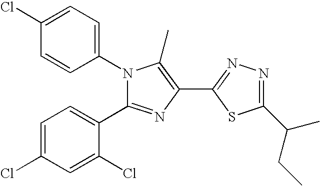 | 2-sec-Butyl-5-(1-(4-chlorophenyl)-2-(2,4-dichlorophenyl)-5-methyl-1H-imidazol-4-yl)-1,3,4-thiadiazole | 28.1 |
| 30 | 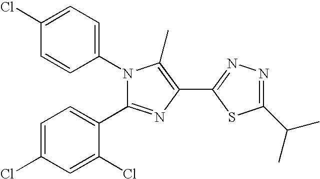 | 2-(1-(4-Chlorophenyl)-2-(2,4-dichlorophenyl)-5-methyl-1H-imidazol-4-yl)-5-isopropyl-1,3,4-thiadiazole | 22.2 |
| 31 | 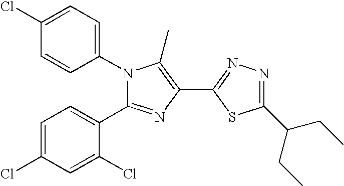 | 2-(1-(4-Chlorophenyl)-2-(2,4-dichlorophenyl)-5-methyl-1H-imidazol-4-yl)-5-(pentan-3-yl)-1,3,4-thiadiazole | 19.7 |
| 32 | 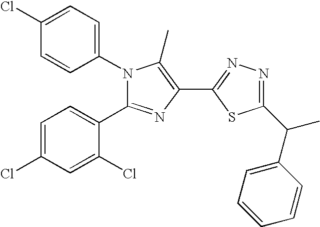 | 2-(1-(4-Chlorophenyl)-2-(2,4-dichlorophenyl)-5-methyl-1H-imidazol-4-yl)-5-(1-phenylethyl)-1,3,4-thiadiazole | 16.9 |
| 33 | 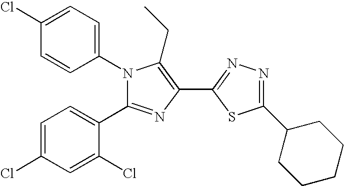 | 2-(1-(4-Chlorophenyl)-2-(2,4-dichlorophenyl)-5-ethyl-1H-imidazol-4-yl)-5-cyclohexyl-1,3,4-thiadiazole | — |
| 34 | 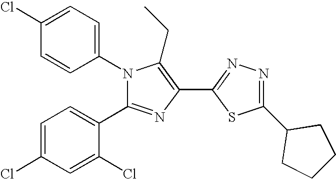 | 2-(1-(4-Chlorophenyl)-2-(2,4-dichlorophenyl)-5-ethyl-1H-imidazol-4-yl)-5-cyclopentyl-1,3,4-thiadiazole | 17.6 |
| 35 | 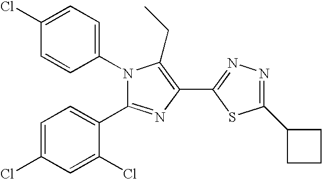 | 2-(1-(4-Chlorophenyl)-2-(2,4-dichlorophenyl)-5-ethyl-1H-imidazol-4-yl)-5-cyclobutyl-1,3,4-thiadiazole | 16.1 |
| 36 | 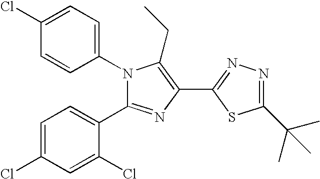 | 2-tert-Butyl-5-(1-(4-chlorophenyl)-2-(2,4-dichlorophenyl)-5-ethyl-1H-imidazol-4-yl)-1,3,4-thiadiazole | 15.8 |
| 37 | 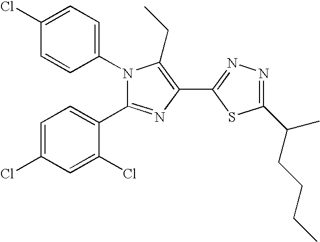 | 2-(1-(4-Chlorophenyl)-2-(2,4-dichlorophenyl)-5-ethyl-1H-imidazol-4-yl)-5-(hexan-2-yl)-1,3,4-thiadiazole | 8.05 |
| 38 | 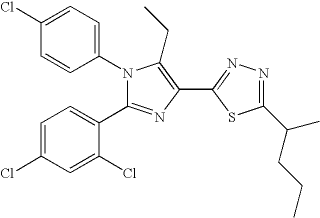 | 2-(1-(4-Chlorophenyl)-2-(2,4-dichlorophenyl)-5-ethyl-1H-imidazol-4-yl)-5-(pentan-2-yl)-1,3,4-thiadiazole | 12.9 |
| 39 | 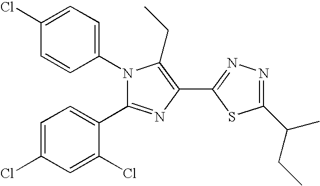 | 2-sec-Butyl-5-(1-(4-chlorophenyl)-2-(2,4-dichlorophenyl)-5-ethyl-1H-imidazol-4-yl)-1,3,4-thiadiazole | 8.46 |
| 40 | 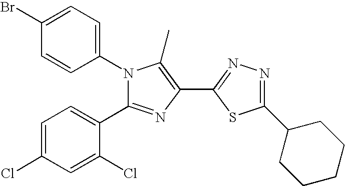 | 2-(1-(4-Bromophenyl)-2-(2,4-dichlorophenyl)-5-methyl-1H-imidazol-4-yl)-5-cyclohexyl-1,3,4-thiadiazole | 56.6 |
| 41 | 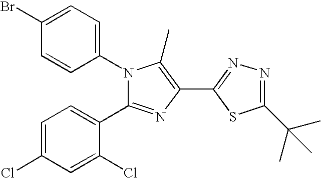 | 2-(1-(4-Bromophenyl)-2-(2,4-dichlorophenyl)-5-methyl-1H-imidazol-4-yl)-5-tert-butyl-1,3,4-thiadiazole | 3.45 |
| 42 | 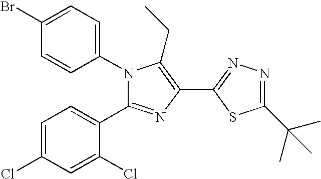 | 2-(1-(4-Bromophenyl)-2-(2,4-dichlorophenyl)-5-ethyl-1H-imidazol-4-yl)-5-tert-butyl-1,3,4-thiadiazole | 1.91 |
| 43 | 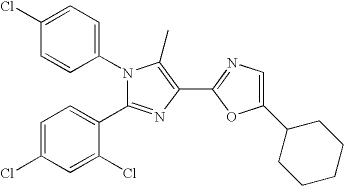 | 2-(1-(4-Chlorophenyl)-2-(2,4-dichlorophenyl)-5-methyl-1H-imidazol-4-yl)-5-cyclohexyloxazole | 78.6 |
| 44 | 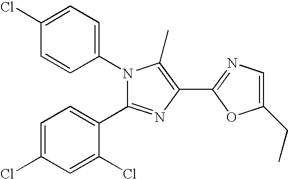 | 2-(1-(4-Chlorophenyl)-2-(2,4-dichlorophenyl)-5-methyl-1H-imidazol-4-yl)-5-ethyloxazole | 84.0 |
| 45 | 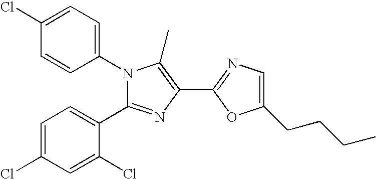 | 5-Butyl-2-(1-(4-chlorophenyl)-2-(2,4-dichlorophenyl)-5-methyl-1H-imidazol-4-yl)oxazole | 11.6 |
| 46 | 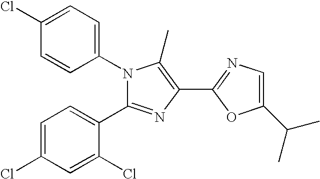 | 2-(1-(4-Chlorophenyl)-2-(2,4-dichlorophenyl)-5-methyl-1H-imidazol-4-yl)-5-isopropyloxazole | 26.4 |
| 47 | 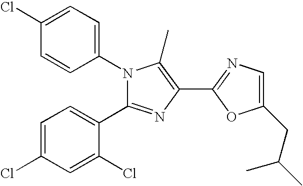 | 2-(1-(4-Chlorophenyl)-2-(2,4-dichlorophenyl)-5-methyl-1H-imidazol-4-yl)-5-isobutyloxazole | 13.1 |
| 48 | 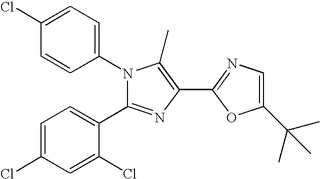 | 2-tert-Butyl-5-(1-(4-chlorophenyl)-2-(2,4-dichlorophenyl)-5-methyl-1H-imidazol-4-yl)oxazole | 44.4 |
| 49 | 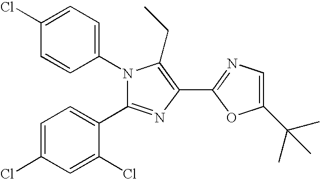 | 2-tert-Butyl-5-(1-(4-chlorophenyl)-2-(2,4-dichlorophenyl)-5-ethyl-1H-imidazol-4-yl)oxazole | 32.2 |
| 50 | 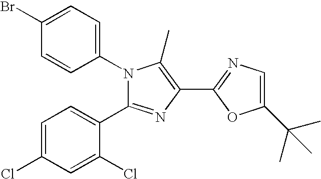 | 2-(1-(4-Bromophenyl)-2-(2,4-dichlorophenyl)-5-methyl-1H-imidazol-4-yl)-5-tert-butyloxazole | 16.9 |
| 51 | 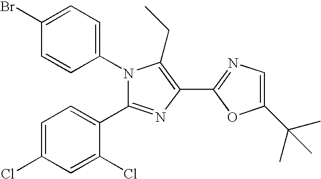 | 2-(1-(4-Bromophenyl)-2-(2,4-dichlorophenyl)-5-ethyl-1H-imidazol-4-yl)-5-tert-butyloxazole | 8.98 |
| 52 | 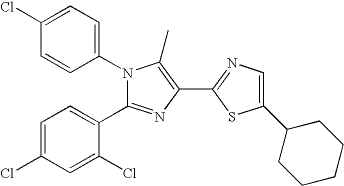 | 2-(1-(4-chlorophenyl)-2-(2,4-dichlorophenyl)-5-methyl-1H-imidazol-4-yl)-5-cyclohexylthiazole | 74.5 |
| 53 | 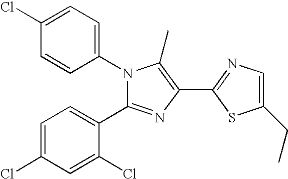 | 2-(1-(4-Chlorophenyl)-2-(2,4-dichlorophenyl)-5-methyl-1H-imidazol-4-yl)-5-ethylthiazole | 70.2 |
| 54 | 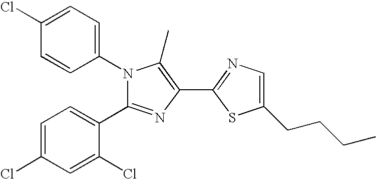 | 5-Butyl-2-(1-(4-chlorophenyl)-2-(2,4-dichlorophenyl)-5-methyl-1H-imidazol-4-yl)thiazole | 63.3 |
| 55 | 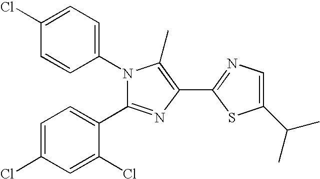 | 2-(1-(4-Chlorophenyl)-2-(2,4-dichlorophenyl)-5-methyl-1H-imidazol-4-yl)-5-isopropylthiazole | 16.0 |
| 56 | 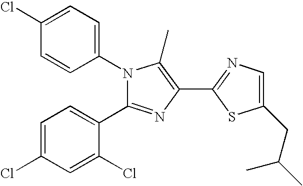 | 2-(1-(4-Chlorophenyl)-2-(2,4-dichlorophenyl)-5-methyl-1H-imidazol-4-yl)-5-isobutylthiazole | 46.4 |
| 57 | 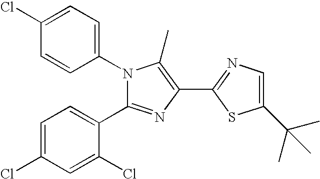 | 2-tert-Butyl-5-(1-(4-chlorophenyl)-2-(2,4-dichlorophenyl)-5-methyl-1H-imidazol-4-yl)thiazole | 45.3 |
| 58 | 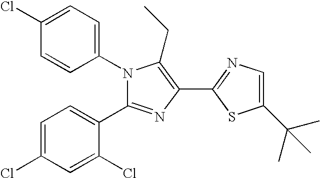 | 2-tert-Butyl-5-(1-(4-chlorophenyl)-2-(2,4-dichlorophenyl)-5-ethyl-1H-imidazol-4-yl)thiazole | 26.0 |
| 59 | 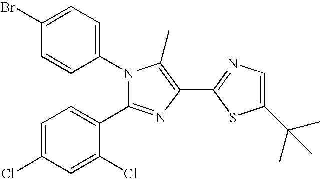 | 2-(1-(4-Bromophenyl)-2-(2,4-dichlorophenyl)-5-methyl-1H-imidazol-4-yl)-5-tert-butylthiazole | 16.0 |
| 60 | 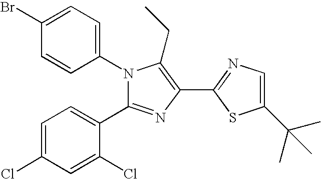 | 2-(1-(4-Bromophenyl)-2-(2,4-dichlorophenyl)-5-ethyl-1H-imidazol-4-yl)-5-tert-butylthiazole | 10.5 |

| | | |
|---|---|---|
| Control compound | 2-(1-(2-Chlorophenyl)-5-(4-chlorophenyl)- | 79 |
| | 4-methyl-1H-pyrazol-3-yl)-4-((6- | |
| | methylpyridin-2-yl)methyl)morpholine | |

As shown in Table 1, the inventive compounds are effective as a cannabinoid CB₁ receptor inverse agonist or antagonist.

While the invention has been described with respect to the specific embodiments, it should be recognized that various modifications and changes may be made by those skilled in the art to the invention which also fall within the scope of the invention as defined as the appended claims.

What is claimed is:

1. A compound of formula (I) or a pharmaceutically acceptable salt thereof: 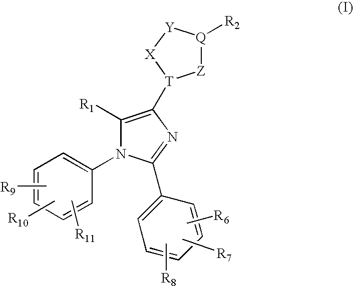
wherein:
R₁ is hydrogen, C₁₋₅ alkyl, substituted C₁₋₅ alkyl, C₂₋₄ alkenyl, substituted C₂₋₄ alkenyl, C₂₋₄ alkynyl, substituted C₂₋₄ alkynyl, or (CH₂)ₙ—C₃₋₅ carbocycle, n being 0 or 1;
R₂ is hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2;
R₃ and R₄ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or
R₃ and R₄, together with the nitrogen atom to which they are bonded, form a 4- to 10-membered saturated or unsaturated heterocyclic ring which is optionally substituted by one or more C₁₋₃ alkyl, benzyl, phenyl, C₁₋₃ alkoxy or halogen;
R₅ is phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substitutes;
R₆, R₇, R₈, R₉, R₁₀ and R₁₁ are each independently hydrogen, halogen, C₁₋₃ alkyl, C₁₋₃ alkoxy or trifluoromethyl;
X, Y and Z are each independently selected from the group consisting of —C(R₁₂)═, —O—, —N═, —N(R₁₃)— and —S— to form an aromatic heterocycle together with Q and T;
Q and T are each independently 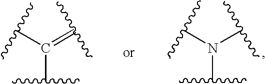
with the proviso that both Q and T can not be simultaneously 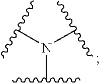
and
R₁₂ and R₁₃ are each independently hydrogen, carbocycle, substituted carbcycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, C₂₋₆ alkynyl optionally substituted by alkoxy or halogen, (CH₂)ₘ—C₃₋₆ carbocycle optionally substituted by alkoxy or halogen, or (CH₂)ₘ—R₅, m being 1 or 2, and R₅ having the same meaning as defined above.

2. The compound of claim 1, which is a compound of formula (Ia), (Ib), (Ic) or (Id): 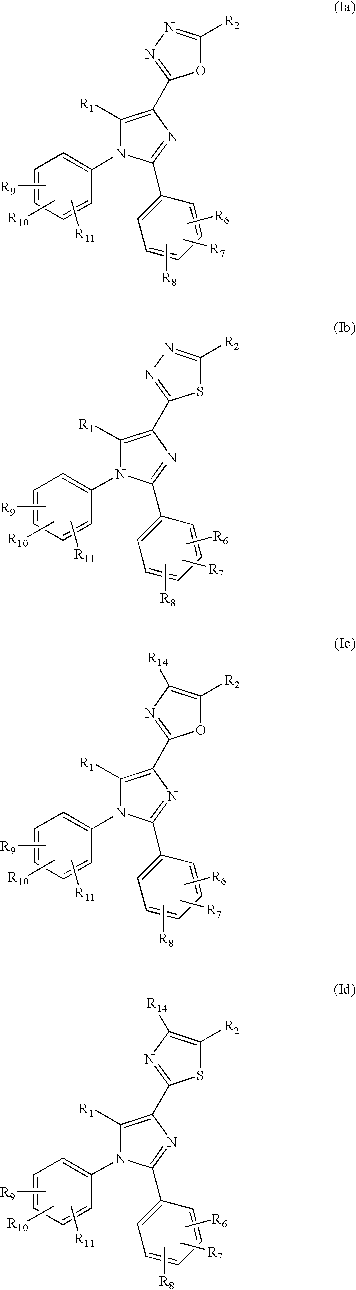
wherein, R₁, R₂, R₆, R₇, R₈, R₉, R₁₀ and R₁₁ have the same meanings as defined in claim 1; and R₁₄ has the same meaning as defined for R₂; or R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

3. A method for preparing the compound of formula (Ia) of claim 2, which comprises (i) reacting a carboxylic acid derivative of formula (6) with a hydrazide compound of formula (7) in the presence of a coupling reagent in a solvent, and (ii) cyclizing the resulting product using a dehydrating agent:

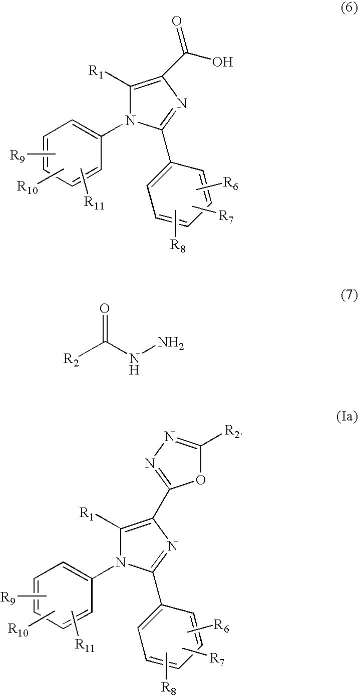

4. The method of claim 3, wherein the dehydrating agent used in (ii) is Burgess reagent.

5. A method for preparing the compound of formula (Ib) of claim 2, which comprises (i) reacting a carboxylic acid derivative of formula (6) with a hydrazide compound of formula (7) in the presence of a coupling reagent in a solvent and (ii) cyclizing the resulting product using Lawesson's reagent: 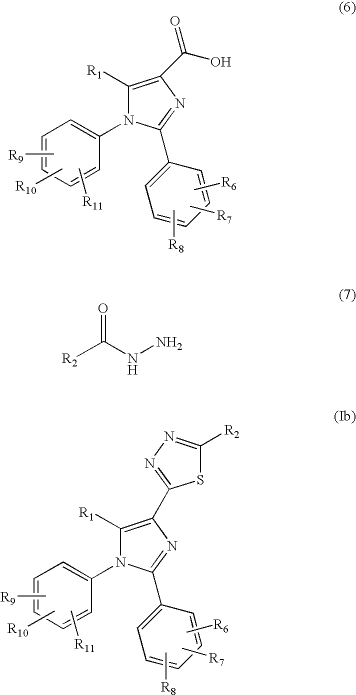
wherein, R₁, R₂, R₆, R₇, R₈, R₉, R₁₀ and R₁₁ have the same meanings as defined in claim 1.

6. A method for preparing the compound of formula (Ic) of claim 2, which comprises (i) reacting a carboxylic acid derivative of formula (6) with a salt of an aminoketone compound of formula (9) in the presence of a coupling reagent in a solvent, (ii) cyclizing the resulting product using a dehydrating agent: 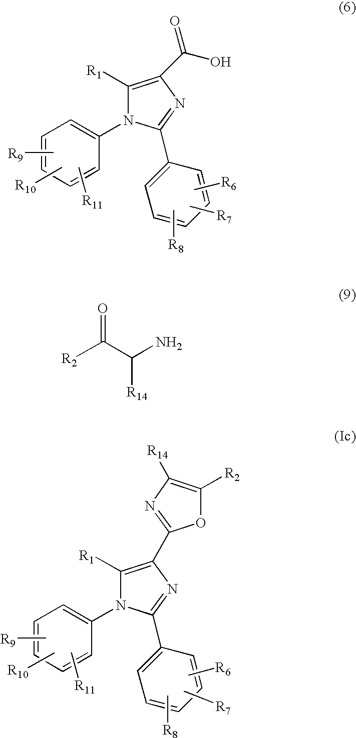
wherein, R₁, R₂, R₆, R₇, R₈, R₉, R₁₀ and R₁₁ have the same meanings as defined in claim 1; and R₁₄ has the same meaning as defined for R₂; or R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

7. The method of claim 6, wherein the dehydrating agent used in (ii) is Burgess reagent.

8. A method for preparing the compound of formula (Id) of claim 2, which comprises (i) reacting a carboxylic acid derivative of formula (6) with a salt of an aminoketone compound of formula (9) in the presence of a coupling reagent in a solvent and (ii) cyclizing the resulting product using Lawesson's reagent: 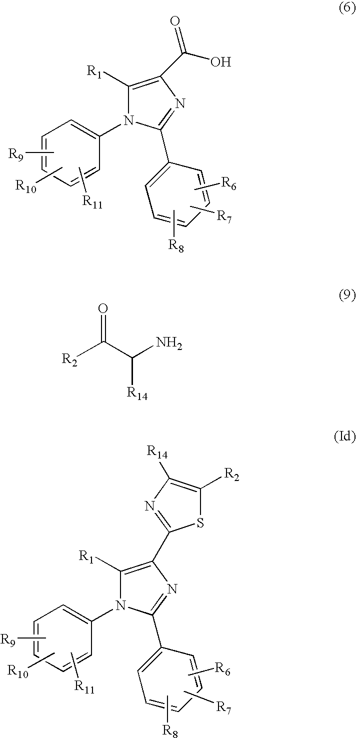
wherein, R₁, R₂, R₆, R₇, R₈, R₉, R₁₀ and R₁₁ have the same meanings as defined in claim 1; and R₁₄ has the same meaning as defined for R₂; or R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

9. A pharmaceutical composition comprising the compound of formula (I) of claim 1 as an active ingredient and a pharmaceutically acceptable carrier.

10. A method for inhibiting cannabinoid CB₁ receptor in a mammal, which comprises administering the compound of formula (I) of claim 1 to the mammal.

11. A method for preventing or treating obesity and obesity-related metabolic disorders in a mammal, which comprises administering the compound of formula (I) of claim 1 to the mammal.
